(19) United States
(12) Patent Application Publication
(10) Pub. No.: US 2006/0167271 A1
(43) Pub. Date: Jul. 27, 2006

(54) Purified compounds that inhibit intracellular alphax4/paxillin binding

(76) Inventors: Mark Ginsberg, San Diego, CA (US); Dale Boger, LaJolla, CA (US)

Correspondence Address: WELSH & KATZ, LTD, 120 S RIVERSIDE PLAZA, 22ND FLOOR, CHICAGO, IL, 60606, US

(21) Appl. No.: 10/535,017

(22) PCT Filed: Oct. 30, 2003
(86) PCT No.: PCT/US03/34460

§ 371 Date: May 12, 2005

Related U.S. Application Data
(60) Provisional application No. 60/426,436, filed on Nov. 14, 2002.

(51) Int. Cl. A61K 38/06; C07D 487/02

(52) U.S. Cl. 548/429; 530/331

(57) ABSTRACT

A purified compound and its pharmaceutically acceptable salt that inhibits the binding between the integrin intracellular or cytoplasmic tail polypeptide and Paxillin, a pharmaceutical composition containing that compound or salt and a method of treating a biological function in an animal using that compound or salt are disclosed. The purified compound corresponds in structure to Formula I, 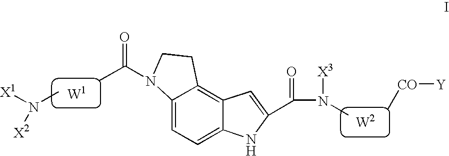
wherein W¹ and W², X¹, X² and X³ and Y are defined within.

### GOVERNMENTAL SUPPORT

The present invention was made with the financial support of the National Institutes of Health under contracts CA78045, AR27214, and HL48728. The U.S. government has certain rights in this invention.

### BACKGROUND ART

The integrin α₄β₁ (also know as VLA-4, very late antigen 4) is a cell surface receptor that plays an important role in embryogenesis, hematopoiesis, and the immune response [Stewart et al. (1995) Curr. Opin. Cell Biol. 7, 690-696; and Shimizu et al. (1999) Adv. Immunol. 72, 325-380]. That protein binds natural ligands including vascular cell adhesion molecule 1 (VCAM-1) and an alternatively spliced connecting segment (CS-1) from the extracellular matrix protein, fibronectin. It mediates cellular adhesion and activation through a variety of cell-cell and cell-matrix interactions that regulate leukocyte migration into tissues during inflammatory responses and lymphocyte trafficking [Springer (1994) Cell 76, 301-314; and Lobb et al. (1994) J. Clin. Invest. 94, 1722-1728].

This integrin is believed to regulate cellular functions differently from other integrins because the α4 cytoplasmic tail binds tightly to the signaling adaptor protein Paxillin through a short conserved sequence motif dominated by two residues (Glu⁹⁸³ and Tyr⁹⁹¹) [Hemler et al. (1992) Cold Spring Harbor Symp. Quant. Biol. 57, 213-220; and Liu et al. (2000) J. Biol. Chem. 275, 22736-227421. The α4/Paxillin interaction leads to enhanced rates of cell migration and reduced rates of cell spreading, focal adhesion, and stress fiber formation (FIG. 1) [Liu et al. (1999) Nature 402, 676-681]. Similarly, the intracellular α9 polypeptide portion of the α₉β₁ integrin also binds Paxillin and is involved in extravasation of neutrophils at sites of acute inflammation. [See, Shouchun et al. (2001) J. Biol. Chem. 276(40):37086-37092.] These biological responses to integrin-mediated cell adhesion contribute to leukocyte migration and changes in gene expression important in chronic inflammation. See, also Ginsberg et al. WO 00/73342, published 7 Dec. 2000.

Although extracellular inhibitors for this class of integrins are under development for the treatment of asthma and multiple sclerosis [Ho et al. (2000) J. Org. Chem. 65, 6743-6748; Tilley et al. (2000) Bioorg. Med. Chem. Lett. 11, 1-4; Lobb et al. (1996) Eur. Respir. J. Suppl. 9, 104s-108s; Vanderslice et al. (1997) J. Immunol. 158, 1710-1718; Jackson et al. (1997) J. Med. Chem. 40, 3359-3368; and Lin et al. (1998) Curr. Opin. Chem. Biol. 2, 453-457], inhibitors targeting the unique cytoplasmic Paxillin/α4 or Paxillin/α9 interaction have not yet been described or explored. Such intracellular versus extracellular inhibitors offer a potential opportunity for the development of compounds with distinct therapeutic profiles, would constitute a rare example of small molecule therapeutic intervention through disruption of a protein-protein interaction, and could ultimately lead to new treatments for diseases including asthma, multiple sclerosis, and rheumatoid arthritis.

Because the structural basis of the Paxillin/α4 and Paxillin/α4 interactions are unknown, a combinatorial chemistry approach was undertaken in search of small molecule antagonists of this protein-protein interaction [Floyd et al. (1999) Prog. Med. Chem. 36, 91-168; and Toogood (2002) J. Med. Chem. 45, 1543-1559]. Our efforts in this area have focused on the exploitation of a technically nondemanding solution-phase strategy that dependably delivers pure individual compounds or small to large combinatorial mixtures [Cheng et al. (1996) J. Am. Chem. Soc. 118, 2567-2573; and Boger et al (1996) J. Am. Chem. Soc. 118, 2109-2110].

A number of such libraries were recently reported (Boger et al (1998) Bioorg. Med. Chem. 6, 1347-1378; Boger et al (1998) Tetrahedron 54, 3955-3970; and Boger et al (1999) J. Org. Chem. 64, 7094-7100], including the solution-phase preparation of a 1000 membered library assembled in two distinct formats: (1) a traditional library composed of 100 mixtures of 10 compounds [Boger et al. (2000) J. Am. Chem. Soc. 122, 6382-6394], and (2) a technically less demanding positional scanning library [Boger (2000) Bioorg. Med. Chem. 8, 2049-2057].

The synthesis of positional scanning libraries [Houghten et al. (1991) Nature 354, 84-86; Houghten et al. (1992) Biotechniques 13, 412-421; Pinilla et al. (1992) Biotechniques 13, 901-905; Dooley et al. (1993) Life Sci. 52, 1509-1517] represents one of the most useful protocols for mixture synthesis, but can only be conducted with solution-phase techniques and is not easily adapted to solid-phase synthesis. Not only is it much less time intensive than the parallel synthesis of individual compounds or small mixtures and technically less demanding than spatially arrayed [Fodor et al. (1991) Science 251, 767-773] or tagged split-and-mix library synthesis [Furka et al. (1991) Int. J. Pept. Protein Res. 37, 487-493; Sebestyen et al. (1993) Bioorg. Med. Chem. Lett. 3, 413-418; Brenner et al. (1992) Proc. Natl. Acad. Sci. USA 89, 5381-5383; Ohlmeyer et al. (1993) Proc. Natl. Acad. Sci. USA 90, 10922-10926; Kerr et al. (1993) J. Am. Chem. Soc. 115, 2529-2531; Moran et al. (1995) J. Am. Chem. Soc. 117, 10787-10788; Nicolaou et al. (1995) Angew. Chem. Int. Ed. Engl. 34, 2289-2291; and Guiles et al. (1998) Angew. Chem. Int. Ed. 37, 926-928], but it produces depository libraries for use in multiple screens and capable of immediate deconvolution [Moran et al. (1995) J. Am. Chem. Soc. 117, 10787-10788; Nicolaou et al. (1995) Angew. Chem. Int. Ed. Engl. 34, 2289-2291; Guiles et al. (1998) Angew. Chem. Int. Ed. 37, 926-928; Geysen et al. (1986) Mol. Immunol. 23, 709-715; Erb et al. (1994) Proc. Natl. Acad. Sci. USA 91, 11422-11426; Deprez et al. (1995) J. Am. Chem. Soc. 117, 5405-5406; Boger et al. (1998) J. Am. Chem. Soc. 120, 7220-7225; Schwabacher et al. (1999) J. Am. Chem. Soc. 121, 8669-8670; Eliseev (1998) Curr. Opin. Drug Discov. Dev. 1, 106-115; Lin et al. (1997) J. Am. Chem. Soc. 119, 5249-5250; Hajduk et al. (1997) J. Am. Chem. Soc. 119, 12257, 12261; and Boger et al. (2000) J. Org. Chem. 65, 1467-1474].

Thus, unlike other mixture deconvolution protocols, positional scanning libraries can provide lead identities in a single round of assays. Despite these attributes, it is not clear how well such libraries may perform in screens for inhibition of protein-protein interactions. The disclosure that follows hereinafter provides results of the screening of our libraries enlisting an ELISA assay using the immobilized α4 cytoplasmic tail and examining the inhibition of soluble recombinant Paxillin binding that led to the discovery of the initial class of agents that can disrupt integrin/Paxillin binding.

### BRIEF SUMMARY OF THE INVENTION

The present invention contemplates a purified compound and its pharmaceutically acceptable salt that inhibits the binding between an integrin intracellular (cytoplasmic) tail polypeptide such as the α4 or α9 polypeptide and Paxillin, a pharmaceutical composition containing that compound or salt and a method of treating an animal such as a mammal having a biological function that is mediated by integrin/Paxillin binding such as inflammation using that compound or salt. More specifically, the present invention contemplates a purified compound whose structure corresponds to Formula I, or a pharmaceutically acceptable salt of that purified compound

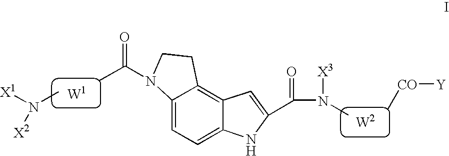

wherein the structures W¹ and W² are the same or different and are a ring system containing one, two or three five-, six- or seven-membered rings of which at least one is aromatic.

X¹ and X² are independently hydrido or R¹—C(O), R¹—NHC(O), or R¹—NHC(S), and R¹ is hydrido or ZQ, or X² is a bond between the depicted nitrogen atom and the structure W¹ so that the depicted nitrogen atom is a ring atom of structure W¹.

X³ is hydrido or X³ is a bond between the depicted nitrogen atom and the structure W² so that the depicted nitrogen atom is a ring atom of structure W².

Z is amino wherein the amino nitrogen is (i) unsubstituted, or (ii) substituted with one or two substituents containing a total of up to fourteen atoms that are carbon, nitrogen, oxygen or sulfur that are independently selected from the group consisting of an alkyl, alkenyl, alkynyl, aryl, heteroaryl, aralkyl, cycloalkyl, heterocyclo, and a heterocycloalkyl group, or (iii) wherein the amino nitrogen and two substituents attached thereto form a 5- to 8-membered heterocyclo or heteroaryl ring containing zero to two additional heteroatoms that are nitrogen, oxygen or sulfur.

Q is a hydrocarbyl group containing two to about ten carbon atoms.

Preferred compounds of Formula I that correspond in structure to Formulas II and III are also contemplated, as are their pharmaceutically acceptable salts.

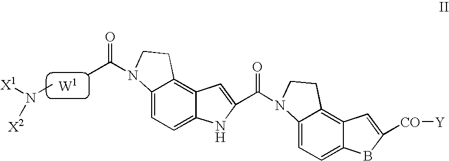

wherein B is O, NH or S, and each of W¹, X¹, X² and Y is as defined before.

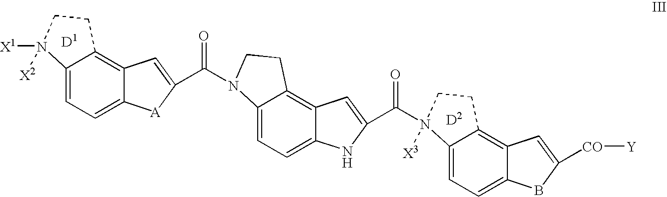

wherein each of A and B is independently O, NH or S;

dotted portions of structures D¹ and D² are independently present or absent such that when either is present, the corresponding X² and X³ substituent is absent;

X¹ and X², when present, are independently hydrido or R¹—C(O), R¹—NHC(O), or R¹—NHC(S), and R¹ is hydrido or ZQ, or when D¹ is present, X² is a bond between the depicted nitrogen atom and the dotted ring structure so that the depicted nitrogen atom is a ring atom of the dotted structure D¹;

X³ is hydrido or is a bond between the depicted nitrogen atom and the dotted structure so that the depicted nitrogen atom is a ring atom of the dotted structure D²; and

R¹, ZQ and Y are as defined before.

Still further compounds and salts are contemplated as is discussed hereinafter.

A pharmaceutical composition is also contemplated. Such a composition contains an effective amount of a purified compound of Formula I or its pharmaceutically acceptable salt dissolved or dispersed in a physiologically acceptable diluent.

A method of treatment is also contemplated. In accordance with a contemplated method, an effective amount of a compound of Formula I or a pharmaceutically acceptable salt of that compound dissolved or dispersed in a physiologically acceptable diluent is administered to an animal such as a mammal having a biological function mediated by intergin/Paxillin binding and is therefore in need of such treatment. Multiple administrations are contemplated in a single day, over several days, several months and several years to alleviate the symptoms of the condition. Illustrative biological functions include inflammation such as inflammatory bowel disease, arthritis, multiple sclerosis and asthma, would healing leading to scarring and atherosclerosis.

### BRIEF DESCRIPTION OF THE DRAWINGS

In the drawings forming a portion of this disclosure,

FIG. 1 is a schematic overview of α4/Paxillin binding-mediated enhancement of cell migration in which Paxillin (Pax) binds to the α4 intracellular cytoplasmic tail of VLA-4 (α₄β₁ integrin). Dimerization of two Pax/VLA-4 complexes occurs through binding to VCAM-1 in the extracellular compartment and triggers a cascade of events and the gene expression responsible for cell migration.

FIG. 2 illustrates the ten aromatic amino acid building blocks of the two libraries used in the studies of the present invention.

FIG. 3 schematically represents the two libraries utilized herein: the 10-Member Mixture Library and the Positional Scanning Library used in the studies of the present invention.

FIG. 4 is a schematic representation of the high throughput screening by ELIZA used herein in which the α4 tail is coated onto a Ni-NTA microtiter plate (a), Paxillin is permitted to bind to α4 (b), first antibody is added (anti-Paxillin) (c), second antibody is added (HRP-conjugated, anti-mouse) (d), and Pax/α4 binding efficiency is measured by detection at 490 nm (e). For binding inhibition, compounds were added during Paxillin incubation (step b). 100% with no Paxillin and no compound, zero % inhibition was measured from incubation with Paxillin and no compound.

FIG. 5 illustrates results obtained in inhibition of α4/Paxillin binding by the Positional Scanning Libraries designated 12-AxBC, 13-AByC, and 14-ABCz. Each mixture was assayed in triplicate at 5, 10, and 20 μM (total compound), and the results are reported as percent inhibition of Paxillin/α4 binding.

FIG. 6 in two parts as 6A and 6B includes results of an affinity chromatography binding study (6A) and scanning densitometry results shown in graphical form for the bound Paxillin (6B). In 6A, recombinant HA-tagged GST-Paxillin (100 nM) was added to Ni²⁺-charged resin loaded with α4 tail protein in the absence (lane a) or presence of compound 11-A7B7C7 (lane b, 5 μM; lane c, 25 μM; lane d, 100 μM) or compound 11-A6B6C6 (lane e, 25 μM). Bound protein was collected and separated by SDS-PAGE under reducing conditions, transferred to a nitrocellulose membrane, and stained with HA-tag-specific antibody, 12CA5. In 6B, bound Paxillin was quantified by scanning densitometry of those immunoblots using the NIH Image program. The depicted results are representative from two studies.

FIG. 7 is a graph that shows the dose-related effect of compound 11-A7B7C7 (closed circles) on integrin α₄β₁-mediated migration of Jurkat T cells, using compound 11-A6B6C6 (open circle) as control. Cell migration was assayed in a modified Boyden chamber assay system as previously described [Rose et al. (2001) J. Immunol. 167, 2824-2830]. In this system, migration is specific to the α4 integrin, as it is completely inhibited by function blocking anti-α4 antibodies [Rose et al. (2001) J. Immunol. 167, 2824-2830]. Transwells (Costar, Corning) polycarbonate membranes containing 3.0 μm pores were coated with 5 μg/ml recombinant soluble VCAM-1. Membranes were blocked with 2% BSA in PBS for 30 minutes at room temperature. 2.0×10⁵ cells in RPMI-1640 containing the indicated concentration of the compound and a final concentration of 0.1% DMSO were added to the top chamber. SDF-1α (R&D Systems) at a final concentration of 15 ng/ml was added to the bottom chamber. Cells were permitted to migrate for 4 hours at 37° C. Cells in the bottom chamber were enumerated with a hemocytometer, and data are expressed as percent inhibition of migration. Depicted are the mean and range of duplicate determinations from one of two studies with similar results.

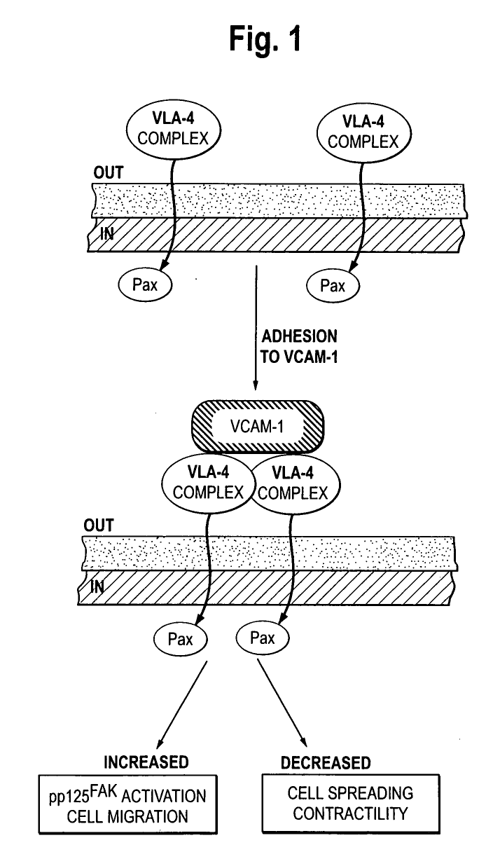

### DETAILED DESCRIPTION OF THE INVENTION

The integrin family of proteins is involved with many biological functions. The cytoplasmic tail polypeptide of integrins α4, α9 and the like is involved in several biological functions including leukocyte migration and trafficking, atherosclerosis and monocyte aggregation during wound healing that can lead to scarring. That integrin involvement is mediated by binding between the integrin cytoplasmic tail polypeptide and the protein, Paxillin.

Inhibition of integrin/Paxillin binding can be used for treatment of an animal biological function mediated by that binding. Such treatments include inhibition of leukocyte migration and trafficking and thereby inflammation caused by those leukocytes, as well as inhibition of atherosclerosis, and also scarring that can occur during wound healing. See, Ginsberg et al. WO 00/73342, published on 7 Dec. 2000.

The present invention contemplates a purified compound and its pharmaceutically acceptable salt that inhibit the binding between an integrin cytoplasmic tail polypeptide and Paxillin, a pharmaceutical composition containing that compound and a method of treating an animal's biological function that is mediated by integrin/Paxillin binding using that compound or salt. The α4 integrin cytoplasmic tail polypeptide (usually referred to herein as α4) is used herein as illustrative of the integrin cytoplasmic tails that bind to Paxillin and for simplicity of expression.

More specifically, the present invention contemplates a purified compound whose structure corresponds to Formula I, or a pharmaceutically acceptable salt of that purified compound

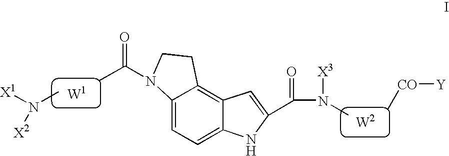

wherein the structures W¹ and W² are the same or different and are a ring system containing one, two or three five-, six- or seven-membered rings of which at least one is aromatic.

X¹ and X² are independently hydrido or R¹—C(O), R¹—NHC(O), or R¹—NHC(S), and R¹ is hydrido or ZQ, or X² is a bond between the depicted nitrogen atom and the structure W¹ so that the depicted nitrogen atom is a ring atom of structure W¹.

X³ is hydrido or X³ is a bond between the depicted nitrogen atom and the structure W² so that the depicted nitrogen atom is a ring atom of structure W².

Z is amino wherein the amino nitrogen is (i) unsubstituted, or (ii) substituted with one or two substituents containing a total of up to fourteen atoms that are carbon, nitrogen, oxygen or sulfur that are independently selected from the group consisting of an alkyl, alkenyl, alkynyl, aryl, heteroaryl, aralkyl, cycloalkyl, heterocyclo, and a heterocycloalkyl group, or (iii) wherein the amino nitrogen and two substituents attached thereto form a 5- to 8-membered heterocyclo or heteroaryl ring containing zero to two additional heteroatoms that are nitrogen, oxygen or sulfur.

Q is a hydrocarbyl group containing two to about ten carbon atoms.

Y is O—R² or Z, wherein R² is hydrido, methyl or Q.

In examining Formula I, above, it is seen that structures W¹ and W² are a ring system containing one, two or three five-, six- or seven-membered rings of which at least one is aromatic. It is preferred that the rings of the ring system be fused rings. Several illustrative aromatic ring systems are illustrated and numbered for convenience below. It is to be understood that the ring systems shown below are illustrative and are not intended to be limiting in that further compounds within the above description are commercially available or are readily prepared by a worker of ordinary skill in this art. Ten of the fourteen ring systems shown below have been used herein as a structure W substituent. Structures W¹ and W² numbered 6, 7, 8, 9, 10, 11 and 12 are preferred for either W¹ or W², with the compound numbered 7 being particularly preferred.

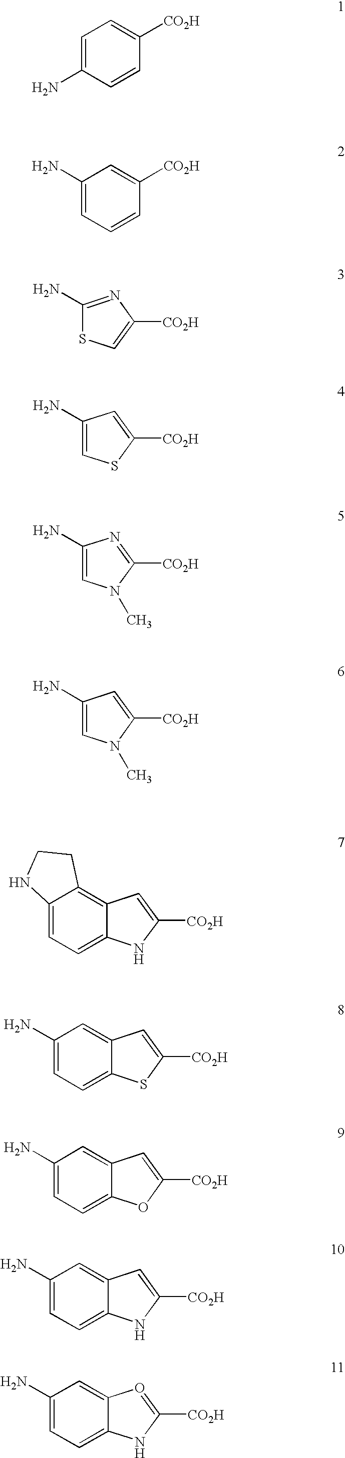

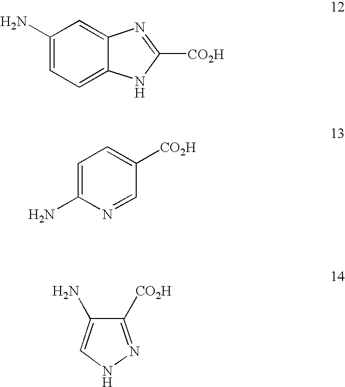

In the depiction of the structure W¹ or W² substituent, it is noted that the bond to the nitrogen atom is shown to be variable. That variation extends from the position of substitution on a ring to the inclusion of the nitrogen atom within the ring structure as is shown for compound 7. It is generally preferred that the bonds to the amino and carboxyl groups be as far apart as possible as seen in compounds 11 and 13, but those bonds can also be adjacent as in compound 14.

The N-terminal nitrogen atom within the ring structure as where X² is a bond between the depicted nitrogen atom and the structure W¹, making that nitrogen have two bonds to the structure W¹. The N-terminal nitrogen atom can also be unsubstituted as where X¹ and X² are both hydrido, or substituted as where one of X¹ and X² is hydrido while the other is an amide [R¹—C(O)], a urea [R¹—NHC(O)] or a thiourea [R¹—NHC(S)] that is unsubstituted where R¹ is hydrido or substituted where R¹ is ZQ.

The “Z” group of ZQ is a nitrogen atom that is (i) unsubstituted as an —NH₂ group, or (ii) monosubstituted or disubstituted with one substituent and one hydrido group or two substituents. The substituents (other than hydrido) can contain up to fourteen atoms that are carbon, nitrogen, oxygen or sulfur. Those one or two substituents are named above and discussed further hereinafter. The “Z” group nitrogen atom can also (iii) join with two other substituents to form an aromatic or aliphatic ring group that contains five, six, seven or eight members and can contain no further hetero, non-carbon, ring atoms, one further non-carbon ring atom or two such heteroatoms.

The “Q” group is a hydrocarbyl group; i.e., a moiety containing only carbon and hydrogen, that contains two through about ten carbon atoms. Hydrocarbyl groups are also discussed hereinafter.

The bonds to the nitrogen from W² are somewhat more limited because that nitrogen links two ring systems. Thus, X³ is hydrido or is a bond between the depicted nitrogen atom and the structure W² so that the depicted nitrogen atom is a ring atom of structure W².

The carboxy-terminus of a contemplated compound can be a carboxyl group as where Y is O—R², where R² is hydrido. The carboxy-terminal moiety can also be an ester as where Y is O—R² and R² is methyl or Q, as defined before. The carboxy-terminal moiety can also be an amide where Y is Z, as also defined above.

One preferred purified compound of Formula I is a purified compound whose structure corresponds to Formula II, below, or its pharmaceutically acceptable salt

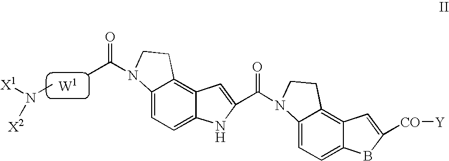

wherein B is O, NH or S, and each of W¹, X¹, X² and Y is as defined before.

Another preferred purified compound of Formula I is a purified compound whose structure corresponds to Formula III, below, or its pharmaceutically acceptable salt

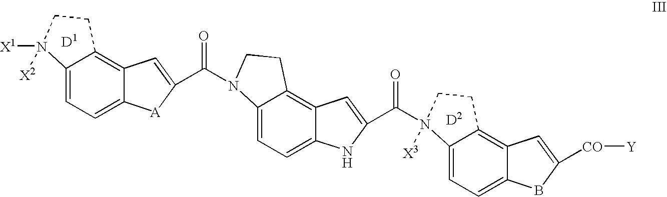

wherein each of A and B is independently O, NH or S;

dotted portions of structures D¹ and D² are independently present or absent such that when either is present, the corresponding X² and X³ substituent is absent;

X¹ and X², when present, are independently hydrido or R¹—C(O), R¹—NHC(O), or R¹—NHC(S), and R¹ is hydrido or ZQ, or when D¹ is present, X² is a bond between the depicted nitrogen atom and the dotted ring structure so that the depicted nitrogen atom is a ring atom of the dotted structure D¹;

X³ is hydrido or is a bond between the depicted nitrogen atom and the dotted structure so that the depicted nitrogen atom is a ring atom of the dotted structure D²; and

R¹, ZQ and Y are as defined before.

A still more preferred purified compound of Formula I is a purified compound whose structure corresponds to Formula IV, below, or its pharmaceutically acceptable salt

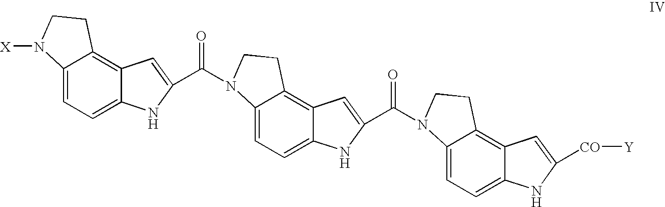

wherein X is R¹—C(O), R¹—NHC(O), or R¹—NHC(S), and R¹ is hydrido or ZQ,

wherein Z is amino wherein the amino nitrogen is (i) unsubstituted, or (ii) substituted with one or two substituents containing a total of up to fourteen atoms that are carbon, nitrogen, oxygen or sulfur and which substituents are independently selected from the group consisting of an alkyl, aryl, heteroaryl, aralkyl, cycloalkyl, aralkoxycarbonyl, alkoxycarbonyl, arylcarbonyl, aralkanoyl, heteroarylcarbonyl and an alkanoyl group, or (iii) wherein the amino nitrogen and two substituents attached thereto form a 5- to 8-membered heterocyclo or heteroaryl ring containing zero to two additional heteroatoms that are nitrogen, oxygen or sulfur, and

Q is a hydrocarbyl group containing two to about ten carbon atoms; and

Y is O—R² or Z,

wherein R² is hydrido, methyl or Q.

Of the above compounds of Formula IV, a compound in which X is hydrido or a 4-(dimethylamino)butyryl group is particularly preferred. In addition, a compound in which Y is O—R² wherein R² is hydrido or methyl is particularly preferred. Illustrative structural formulas for particularly preferred compounds are shown hereinbelow as Formulas A, B and C.

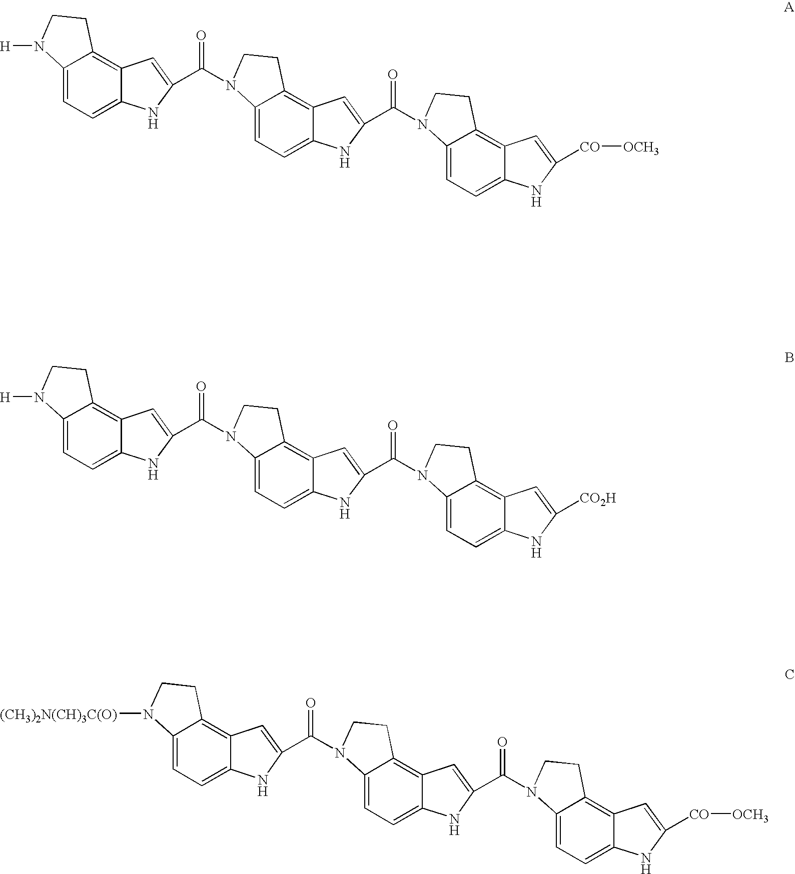

A contemplated compound whose structure corresponds to Formula I or its pharmaceutically acceptable salt typically inhibits binding between integrin (α4) and Paxillin in an amount of at least 50% at a concentration of 5 μM using an in vitro assay discussed hereinafter. More preferably, that inhibition is at least about 60%, and is still more preferably at least about 70%. Most preferably, that inhibition of binding is about 80% or more at 5 μM. Exemplary specific inhibition binding results using integrin α4 are provided hereinafter in Table 1 for 5 μM concentrations, and Tables 2 and 3 for 1 μM concentrations.

As utilized herein, the term “alkyl”, alone or in combination, means a straight-chain or branched-chain alkyl radical containing 1 to about 12 carbon atoms, preferably 1 to about 10 carbon atoms, and more preferably 1 to about 6 carbon atoms. Examples of such radicals include methyl, ethyl, n-propyl, isopropyl, n-butyl, isobutyl, sec-butyl, tert-butyl, pentyl, iso-amyl, hexyl, octyl and the like.

The term “alkenyl”, alone or in combination, means a straight-chain or branched-chain hydrocarbon radical having one or more double bonds and containing 2 to about 12 carbon atoms preferably 2 to about 10 carbon atoms, and more preferably, 2 to about 6 carbon atoms. Examples of suitable alkenyl radicals include ethenyl (vinyl), 2-propenyl, 3-propenyl, 1,4-pentadienyl, 1,4-butadienyl, 1-butenyl, 2-butenyl, 3-butenyl, decenyl and the like.

The term “alkynyl”, alone or in combination, means a straight-chain hydrocarbon radical having one or more triple bonds and containing 2 to about 12 carbon atoms, preferably 2 to about 10 carbon atoms, and more preferably, 2 to about 6 carbon atoms. Examples of alkynyl radicals include ethynyl, 2-propynyl, 3-propynyl, decynyl, 1-butynyl, 2-butynyl, 3-butynyl, and the like.

The word “hydrocarbyl” is used herein as a short hand term to include straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrocarbyl groups, are referred to herein as aryl groups or radicals, as discussed hereinafter. Where a specific aliphatic hydrocarbyl substituent group is intended, that group is recited; i.e., C₁-C₄ alkyl, methyl or dodecenyl. Exemplary hydrocarbyl groups contain a chain of 1 to about 12 carbon atoms, and preferably one to about 10 carbon atoms. A particularly preferred hydrocarbyl group is an alkyl group.

The term “alkoxy”, alone or in combination, means an alkyl ether radical wherein the term alkyl is as defined above. Examples of suitable alkyl ether radicals include methoxy, ethoxy, n-propoxy, isopropoxy, n-butoxy, iso-butoxy, sec-butoxy, tert-butoxy and the like.

The term “cycloalkyl”, alone or in combination, means a cyclic alkyl radical that contains 3 to about 8 carbon atoms. The term “cycloalkylalkyl” means an alkyl radical as defined above that is substituted by a cycloalkyl radical containing 3 to about 8, preferably 3 to about 6, carbon atoms. Examples of such cycloalkyl radicals include cyclopropyl, cyclobutyl, cyclopentyl, cyclohexyl and the like.

A heterocyclic (heterocyclo) or heterocyclo portion of a heterocycloalkyl group or the like is a saturated or partially unsaturated monocyclic, bicyclic or tricyclic heterocycle that contains one or more hetero atoms selected from nitrogen, oxygen and sulfur. Heterocyclo compounds include benzofused heterocyclic compounds such as benzo-1,4-dioxane. Such a moiety can be optionally substituted on one or more ring carbon atoms by halogen, hydroxy, hydroxycarbonyl, alkyl, alkoxy, oxo, and the like, and/or on a secondary nitrogen atom (i.e., —NH—) of the ring by alkyl, aralkoxycarbonyl, alkanoyl, aryl or arylalkyl or on a tertiary nitrogen atom (i.e., ═N—) by oxido and that is attached via a carbon atom. The tertiary nitrogen atom with three substituents can also attached to form a N-oxide [═N(O)—] group.

The term “aryl”, alone or in combination, means a 5- or 6-membered carbocyclic aromatic ring-containing moiety or a fused ring system containing two or three rings that have all carbon atoms in the ring; i.e., a carbocyclic aryl radical. Exemplary carbocyclic aryl radicals include phenyl, indenyl and naphthyl radicals.

The term “heteroaryl”, alone or in combination means a 5- or 6-membered aromatic ring-containing moiety or a fused ring system (radical) containing two or three rings that have carbon atoms and also one or more heteroatoms in the ring(s) such as sulfur, oxygen and nitrogen. Examples of such heterocyclic or heteroaryl groups are pyrrolidinyl, piperidyl, piperazinyl, morpholinyl, thiamorpholinyl, pyrrolyl, imidazolyl (e.g., imidazol-4-yl, 1-benzyloxycarbonylimidazol-4-yl, and the like), pyrazolyl, pyridyl, pyrazinyl, pyrimidinyl, furyl, tetrahydrofuryl, thienyl, triazolyl, tetrazolyl, oxazolyl, oxadiazoyl, thiazolyl, thiadiazoyl, indolyl (e.g., 2-indolyl, and the like), quinolinyl, (e.g., 2-quinolinyl, 3-quinolinyl, 1-oxido-2-quinolinyl, and the like), isoquinolinyl (e.g., 1-isoquinolinyl, 3-isoquinolinyl, and the like), tetrahydroquinolinyl (e.g., 1,2,3,4-tetrahydro-2-quinolyl, and the like), 1,2,3,4-tetrahydroisoquinolinyl (e.g., 1,2,3,4-tetrahydro-1-oxo-isoquinolinyl, and the like), quinoxalinyl, β-carbolinyl, 2-benzofurancarbonyl, benzothiophenyl, 1-, 2-, 4- or 5-benzimidazolyl, and the like radicals.

The term “aralkyl”, alone or in combination, means an alkyl radical as defined above in which one hydrogen atom is replaced by an aryl radical as defined above, such as benzyl, 2-phenylethyl and the like.

The term “aryloxy” means a radical of the formula aryl-O— in which the term aryl has the significance given above. The phenoxy radical is an exemplary aryloxy radical.

The terms “heteroaralkyl” and “heteroaryloxy” mean radicals structurally similar to aralkyl and aryloxy that are formed from heteroaryl radicals. Exemplary radicals include 4-picolinyl and 2-pyrimidinoxy, respectively.

The term “amino”, alone or in combination, means an amine or —NH₂ group whereas the term mono-substituted amino, alone or in combination, means a substituted amine —N(H) (substituent) group wherein one hydrogen atom is replaced with a substituent, and disubstituted amine means a —N(substituent)₂ wherein two hydrogen atoms of the amino group are replaced with independently selected substituent groups.

Amines, amino groups and amides are compounds that can be designated as primary (I°), secondary (II°) or tertiary (III°) or unsubstituted, mono-substituted or N,N-disubstituted depending on the degree of substitution of the amino nitrogen. Quaternary amine (ammonium) (IV°) means a nitrogen with four substituents [—N⁺(substituent)₄] that is positively charged and accompanied by a counter ion, whereas N-oxide means one substituent is oxygen and the group is represented as [—N⁺(substituent)₃-O⁻]; i.e., the charges are internally compensated.

Compositions and Methods

A contemplated purified compound can be used as the compound itself, but is typically present and used in the form of a pharmaceutically acceptable salt. The terms “pharmaceutically acceptable” and “physiologically acceptable” are used adjectivally herein to mean that the modified noun is appropriate for use in a pharmaceutical product.

Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, meglumine (N-methylglucamine) ethylenediamine, and procaine.

Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, benzenesulfonic acid and the like. As such, a contemplated compound is often present in the form of an amine salt derived from an inorganic or organic acid. Exemplary acid salts using some of the above acids include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate and undecanoate.

Also, a basic nitrogen-containing group can be quaternized with such agents as lower alkyl (C₁-C₆) halides, such as methyl, ethyl, propyl, and butyl chloride, bromides, and iodides; dialkyl sulfates like dimethyl, diethyl, dibuytl, and diamyl sulfates, long chain (C₈-C₂₀) halides such as decyl, lauryl, myristyl and dodecyl chlorides, bromides and iodides, aralkyl halides like benzyl and phenethyl bromides, and others to provide enhanced water-solubility. Water or oil-soluble or dispersible products are thereby obtained as desired. The salts are formed by combining the basic compounds with the desired acid.

A pharmaceutical composition containing an effective amount of a purified compound of Formula I or its pharmaceutically acceptable salt dissolved or dispersed in a physiologically acceptable diluent is also contemplated. Total daily dose administered to a host mammal in need of treatment for inflammation in single or divided doses of an α4/Paxillin-inhibiting effective amount can be in amounts, for example, of about 0.001 to about 100 mg/kg body weight daily, preferably about 0.001 to about 30 mg/kg body weight daily and more usually about 0.01 to about 10 mg. Dosage unit compositions can contain such amounts or submultiples thereof to make up the daily dose. A suitable dose can be administered, in multiple sub-doses per day. Multiple doses per day can also increase the total daily dose, should such dosing be desired by the person prescribing the drug.

The dosage regimen for treating inflammation, thrombosis or malignancy with a compound and/or composition of this invention is selected in accordance with a variety of factors, including the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized and whether the compound is administered as Part of a drug combination. Thus, the dosage regimen actually employed can vary widely and therefore can deviate from the preferred dosage regimen set forth above.

A compound or its pharmaceutically acceptable salt useful in the present invention can be formulated as a pharmaceutical composition. Such a composition can then be administered orally, parenterally, by inhalation spray, rectally, or topically in dosage unit formulations containing conventional nontoxic pharmaceutically acceptable carriers, adjuvants, and vehicles as desired. Topical administration can also involve the use of transdermal administration such as transdermal patches or iontophoresis devices. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Suppositories for rectal administration of the drug can be prepared by mixing the drug with a suitable nonirritating excipient such as cocoa butter, synthetic mono- di- or triglycerides, fatty acids and polyethylene glycols that are sold at ordinary temperatures but liquid at the rectal temperature and will therefore melt in the rectum and release the drug.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form varies depending upon the mammalian host treated and the particular mode of administration.

A method of treating a biological function mediated by integrin/Paxillin binding comprises administering to an animal in need thereof an effective amount of a compound of Formula I or a pharmaceutically acceptable salt of said compound dissolved or dispersed in a physiologically acceptable diluent. Exemplary biological functions mediated by integrin/Paxillin binding include inflammation such as is present in inflammatory bowel disease, arthritis, multiple sclerosis, neutrophils extravasation and asthma, as well as would healing leading to scarring and atherosclerosis.

Multiple administrations are contemplated for such treatments. Those administrations can occur in a single day, over several days, several months and several years to alleviate the symptoms of the condition mediated by integrin/Paxillin binding.

Illustrative animals treated in accordance with this method include mammals such as companion animals such as dogs, cats and ferrets, laboratory animals such as rabbits, Guinea pigs, mice and rats, and farm animals such as cows, horses, goats, llamas, alpacas, camels and sheep. Of course, primates such as monkeys, apes and humans are also appropriate subjects. Avian animals such as chickens, turkeys, ducks, Guinea fowl, and geese are also contemplated animals to be treated, as are reptiles such as snakes and lizards and amphibians such as frogs and toads as are present in zoos are also contemplated.

The Compounds

The contemplated purified compounds were present as mixtures in a library that had been prepared in two formats. The present study represents a rare case for which the parallel screening of a traditional library of 100 mixtures of 10 compounds was conducted alongside a related positional scanning library such that the results could be compared [Boger et al. (2000) J. Org. Chem. 65, 1467-1474]. Both approaches led to the identification of the same lead compound, demonstrating the power of the positional scanning strategy. In addition, structural features contributing to the observed inhibition of binding between Paxillin and the α4 tail were clear from the initial screening results, and the subsequent examination of key partial structures of the initial leads define a class of potent Paxillin/α4 antagonists. The potent lead structure (IC₅₀=300 nM) was shown to decrease α₄β₁-mediated human Jurkat T cell migration in a dose-dependent manner, validating this new therapeutic target.

Library Composition

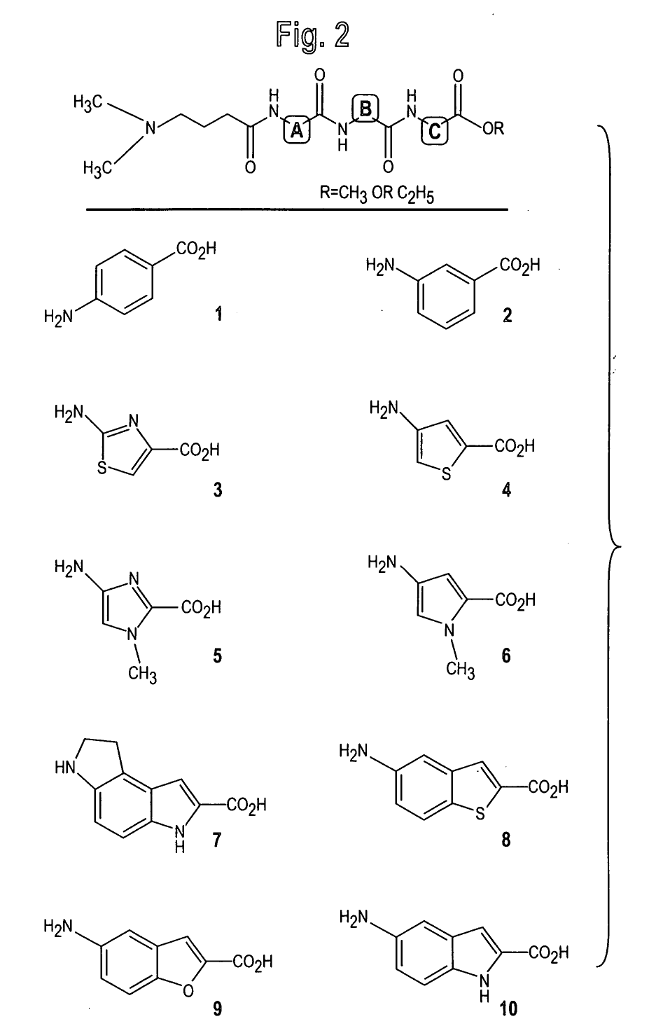

The preparation of a library of 1000 compounds from which the lead emerged in a traditional small mixture format was recently reported [Boger et al., (2000) J. Am. Chem. Soc. 122, 6382-6394]. The structure of the library components subunits (A, B, and C) linked by amide bonds, and a basic side chain 4-(dimethylamino)butyric acid (DMABA) liked to the A subunit (FIG. 2).

Using 10 different aromatic amino acids (FIG. 2), the library was prepared by parallel synthesis of the 100 individual compounds constituting all possible B-C combinations followed by their coupling with the A1-A10 mixture. This provided a 1000-member library in a format of 100 mixtures of 10 compounds (FIG. 2, 11-AByCz).

The solution-phase synthesis of the positional scanning library that contained the same compounds but arranged differently was also reported [Boger et al. (2000) Bioorg. Med. Chem. 8, 2049-2057]. The positional scanning library was comprised of 30 sublibraries that were divided into three sets of ten mixtures each.

Each set is defined by a fixed position of a monomer subunit within the triamide. Within set 1 (FIG. 2, 12-AxBC, A-Scan), subunit (1-10) was individually present at position A, and a full mixture of 1-10 was present at each of positions B and C. In set 2 (13-AByC, B-Scan), the B position is defined with a single subunit, but A and C are undefined (full mixture). In set 3 (14-ABCz, C-Scan), the C position is defined with a single subunit, but A and B are undefined mixtures. The C-terminus of the library compounds was capped as methyl or ethyl esters, and the N-terminus was acylated with 4-(dimethylamino)butyric acid (DMABA).

Screening Results

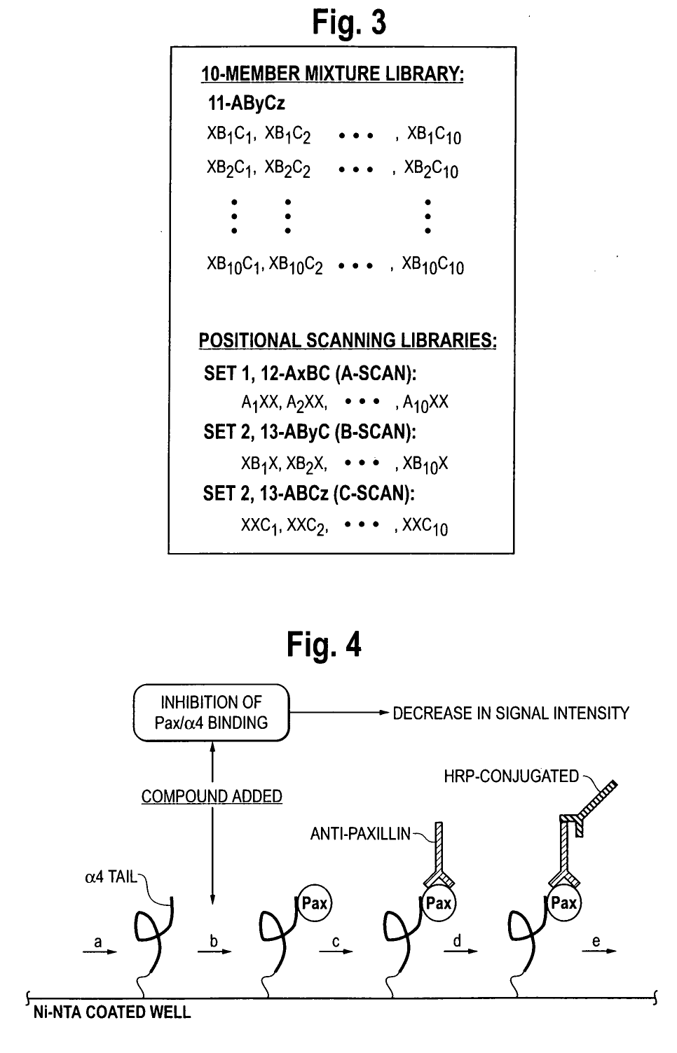

Initially, roughly 40,000 compounds were screened in approximately 2000 wells (most as a mixture of 10 compounds, 50 μM total concentration) in an ELISA assay using an immobilized His-tagged α4 tail [Pfaff et al. (1998) J. Biol. Chem. 273, 6104-6109] and examining the binding inhibition of soluble recombinant Paxillin [Salgia et al. (1995) J. Biol. Chem. 270, 5039-5047] (FIG. 3).

From this screen, mixture library 11-AByCz (50 μM) exhibited numerous mixture hits. This library of 1000 compounds was reassayed at lower concentrations (5 μM total compound, 0.5 μM per component), and the results are reported as percent inhibition versus no compound in Table 1A.

TABLE 1A
Inhibition (%) of α4/Paxillin Binding by
Mixtures 11-AByCz (5 μM total Compound)

| | C1 | C2 | C3 | C4 | C5 | C6 | C7 | C8 | C9 | C10 |
|---|---|---|---|---|---|---|---|---|---|---|
| 11-AB1Cn | 17 | 8 | 21 | 15 | 14 | 22 | 27 | 6 | 2 | 18 |
| 11-AB2Cn | 6 | 11 | 12 | 6 | 23 | 23 | 32 | 9 | 7 | 6 |
| 11-AB3Cn | 2 | 14 | 9 | 17 | 3 | 15 | 24 | 17 | 12 | 21 |
| 11-AB4Cn | 25 | 6 | 4 | 21 | 8 | 8 | 6 | 15 | 6 | 13 |
| 11-AB5Cn | 24 | 3 | 15 | 19 | 11 | 16 | 19 | 8 | 16 | 12 |
| 11-AB6Cn | 19 | 18 | 14 | 11 | 3 | 6 | 9 | 15 | 11 | 16 |
| 11-AB7Cn | 53 | 61 | 60 | 58 | 28 | 78 | 88 | 72 | 71 | 80 |
| 11-AB8Cn | 3 | 0 | 2 | 5 | 16 | 5 | 36 | 0 | 8 | 14 |
| 11-AB9Cn | 25 | 17 | 19 | 22 | 16 | 32 | 38 | 19 | 32 | 45 |
| 11-AB10Cn | 21 | 26 | 24 | 23 | 12 | 31 | 16 | 21 | 75 | 23 |

As is seen, mixtures 11-AB7C6, 11-AB7C7, 11-AB7C10, and 11-AB10C9 exhibited exceptional and consistent activity in this concentration range exhibiting approximately 80% inhibition. In nearly each case, mixture B7 and to a lesser extent C7 mixtures exhibited the most potent inhibition, followed by B10 or C10 mixtures.

The presence of subunits 7 or 10 (two very close structures, see FIG. 2) in each of the four most active mixtures suggested a specific structure-related inhibition. Consequently, each individual compound of the four most potent mixtures was resynthesized from the archived Boc-B7C6, Boc-B7C7, Boc-B7C10, and Boc-B10C9 precursors using the route previously disclosed [Boger et al., (2000) J. Am. Chem. Soc. 122, 6382-6394]. The forty individual compounds were assayed at 1 μM, and the results (% inhibition) are reported in Table 1B, below.

TABLE 1B
Inhibition (%) of α4/Paxillin Binding by
Deconvoluted Samples of 11-AxByCz (1 μM)

| | A1 | A2 | A3 | A4 | A5 | A6 | A7 | A8 | A9 | A10 |
|---|---|---|---|---|---|---|---|---|---|---|
| 11-AnB7C6 | 31 | −8 | 29 | 31 | 47 | −11 | 74 | 60 | 60 | 54 |
| 11-AnB7C7 | 70 | 58 | 80 | 76 | 68 | 66 | 76 | 74 | 81 | 63 |
| 11-AnB7C10 | 40 | 4 | 54 | 54 | 27 | 29 | 77 | 59 | 67 | 54 |
| 11-AnB10C9 | 29 | −9 | 72 | −3 | 2 | −23 | 63 | 48 | −10 | 35 |

Impressively, every molecule derived from mixture 11-AB7C7 was found to be active at this concentration. Except for the closely related 11-A7B10C9, the leads share an identical scaffold composed of two CDPI subunits (11-AxB7C7 or 11-A7B7Cz) and led to the conclusion that the CDPI₂ motif is key to Paxillin/α4 binding inhibition. It was determined that none of the lead compounds exhibited its activity by disrupting the binding of α4 to the Ni-NTA well. We measured (anti-α4) the concentration of α4 integrin bound to the plate in the presence or absence of compound and found that none of the lead compounds destabilized the bound α4.

At the time the initial screening was conducted, the Boc-protected precursors [Boger et al., (2000) J. Am. Chem. Soc. 122, 6382-6394] to the 11-AByCz library (1000 compounds) lacking the DMABA side chain were also examined as identical mixtures of 10 compounds (at concentrations of 50 and 5 μM). Although none of these derivatives was as active as the DMABA derivatives, they did display analogous trends with B7 and C7 mixtures exhibiting the greatest binding inhibition. These results suggested that either the DMABA basic side chain was contributing productively to the binding inhibition or that an N-terminus Boc group was detrimental.

The most active thirteen individual compounds in Table 1B were assayed further at a range of concentrations, and the results are reported as IC₅₀ values in Table 1C, below.

TABLE 1C
Inhibition of α4/Paxillin Binding by Deconvoluted
Samples of 11-AxByCz (IC₅₀ in μM)

| | A1 | A2 | A3 | A4 | A5 | A6 | A7 | A8 | A9 | A10 |
|---|---|---|---|---|---|---|---|---|---|---|
| 11-AnB7C6 | — | — | — | — | — | — | 0.5 | — | — | — |
| 11-AnB7C7 | 0.6 | 1.4 | 0.4 | 0.4 | 0.5 | 2.2 | 0.3 | 0.7 | 0.4 | 4.8 |
| 11-AnB7C10 | — | — | — | — | — | — | 0.4 | — | — | — |
| 11-AnB10C9 | — | — | — | — | — | — | 2.2 | — | — | — |

Compound 11-A7B7C7 that contained three CDPI subunits was the most effective inhibitor of Paxillin/α4 binding, with a IC₅₀ of 300 nM. However, the fact that every component of mixture 11-AB7C7 (11-A1B7C7 to 11-A10B7C7) exhibited such similar activity suggests either that the A subunit is unnecessary or that the binding site is able to accommodate a wide variety of groups at position A.

An important feature in this work rested with the parallel screening of the related positional scanning library for which every component contained in the traditional compound library (11-AByCz) was also present, but assembled such that immediate deconvolution is possible. This library was initially screened at the 50 μM total compound concentration and, like the traditional library, displayed numerous active mixtures. Consequently, the compounds were rescreened at 5, 10, and 20 μM total compound concentrations.

Thus, scanning for the best subunit at position A by screening sublibrary 12-AxBc showed that the greatest inhibition was observed for mixture 12-A7BC at each concentration, identifying the CDPI subunit as the best subunit at position A (FIG. 4). Using the same procedure, CDPI was also identified as the most effective subunit at positions B and C (FIG. 4).

Immediate deconvolution of the results identifies the A7B7C7 combination as a potent Paxillin/α4 binding antagonist. Thus, the same lead compound (11-A7B7C7) was identified from the two different combinatorial strategies. This success is tempered by the fact that the positional scanning library screening and direct deconvolution do not identify as candidate inhibitors 11-A3B7C7, 11-A9B7C7, or 11-A7B7C6, which were identified from the traditional library. That result is a natural consequence of assaying larger 100-compound mixtures and the relative insensitivity of the assay to the contribution of any single, uniquely acting compound in the mixture.

Thus, the global observations were effectively detected with the positional scanning library, and a useful lead structure with defined properties was identified. However, more subtle discoveries within the library were not identified. Thus, the disadvantages associated with the loss of their detection and this information contained within the library is balanced against the advantages of the ease of synthesis of the parent libraries and judged in light of the objectives of the library screening. As in this case, the positional scanning libraries typically would be most effective for lead identification and would be less suitable for lead optimization.

Further Structure-Activity Studies

Following the identification of 11-A7B7C7 by two different combinatorial strategies, the structural features contributing to its properties were defined by examining key partial structures (minimal structure scanning). Notably, a first level structure-activity relationship study was available from the original library screening, albeit conducted on mixtures, indicating that the CDPI subunit (7) was more effective than the closely related benzothiophene, benzofuran, or indole subunits (8-10), with the more closely related indole 10 typically being better than subunits 8 or 9 (i.e., typically: 7>10>9>8).

Consequently, the subsequent studies focused on 13 additional CDPI derivatives containing one, two, or three CDPI subunits. Within each series, the new analogs contained either a methyl ester or a free acid at C-terminal position, and either a dimethylaminobutyric acid (DMABA), a Boc, or a free amine at N-terminal position. Previously unreported analogs [Boger (1987) J. Org. Chem. 52, 1521-1530] that contain the DMABA side chain were prepared using traditional Boc-deprotection (4 N HCl-dioxane) and amide coupling with 4-(dimethylamino)-butyric acid effected by 1-[3-dimethylamino)propyl]-3-ethylcarbodiimide hydrochloride and 4-dimethyl-aminopyridine (EDCI/DMAP) in dimethylformamide (DMF).

Each analog was assayed for Pax/α4 inhibition at a single concentration (1 μM), and the most active compounds were further characterized by measuring their IC₅₀ for α4/Pax binding inhibition. The results are summarized in Table 2, below.

TABLE 2
Inhibition of α4/Paxillin Binding by Key
Substructure Analogues of 11-A7B7C7

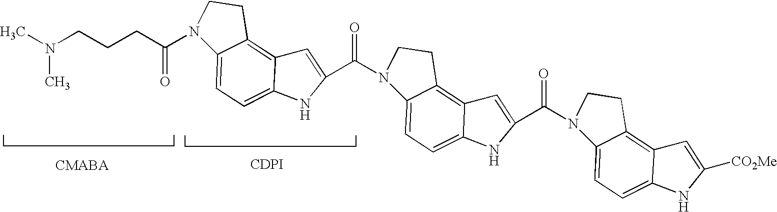

| | Inhibition of | inhibition of |
|---|---|---|
| | Pax/α4 binding | Pax/α4 binding |
| Compound | (% at 1 μM) | (IC₅₀ in μM) |
| Boc-CDPI-OMe, 15 | 3 | nd |
| H-CDPI-OMe, 16 | 19 | nd |
| H-CDPI-OH, 17 | 11 | nd |
| Boc-CDPI-OH, 18 | 6 | nd |
| DMABA-CDPI-OMe, 19 | −3 | nd |
| Boc-CDPI₂-OMe, 20 | 13 | nd |
| H-CDPI₂-OMe, 21 | 14 | nd |
| DMABA-CDPI₂-OMe, 22 | 36 | nd |
| H-CDPI₂-OH, 23 | 27 | nd |
| Boc-CDPI₃-OMe, 24 | 25 | nd |
| H-CDPI₃-OMe, 25 | 73 | 0.3 |
| DMABA-CDPI₃-OMe, | 78 | 0.3 |
| 11-A7B7C7 | | |
| H-CDPI₃-OH, 26 | 68 | 0.5 |

| 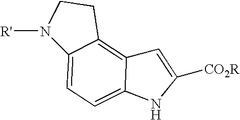 | | | 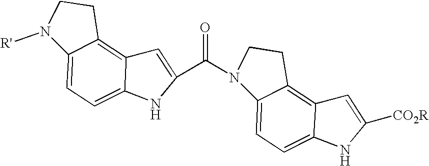 | | | |
|---|---|---|---|---|---|---|
| | | R | R′ | | R | R′ |
| | 15 | Me | Boc | 20 | Me | Boc |
| | 16 | Me | H | 21 | Me | H |
| | 17 | H | H | 22 | Me | DMABA |
| | 18 | H | Boc | 23 | H | H |
| | 19 | Me | DMABA | | | |

Compounds with one CDPI subunit (Compounds 15-19) or two CDPI subunits (Compounds 20-23) were not active or were only weakly active at the concentration tested, and only DMABA-CDPI₂-OMe (Compound 22) exhibited moderate activity (36% inhibition at 1 μM). Moreover, the activity smoothly increased as one progressed through the series CDPI₁<CDPI₂<CDPI₃. Thus, although the nature of the A subunit is not critical to the observed activity, its presence substantially potentiates it.

Reassaying Compound 11-A7B7C7 confirmed its activity (IC₅₀=300 nM) and the analog Compound 25 lacking the basic DMABA side chain exhibited a similar potency (25, IC₅₀=300 nM), indicating that the DMABA group is not necessary for activity. Replacing the DMABA side chain with a Boc group (Boc-CDPI₃-OMe, Compound 24) led to a substantial loss of activity. This observation confirms, as suggested earlier, that the presence of a N-terminus Boc group is detrimental to the inhibition properties.

Just as interestingly, analog Compound 27, lacking both the DMABA group at the N-terminus and the methyl ester at the C-terminus, exhibited only a slight reduction in potency against Paxillin/α4 binding. This result not only indicates that the methyl ester is dispensable, but that the in vivo activity of the leads against the target protein-protein interaction will remain relatively unchanged even upon ester hydrolysis.

Affinity Chromatography Binding Studies

In order to confirm the effectiveness and selectivity of Compound 11-A7B7C7 at inhibiting Paxillin/α4 binding, a series of binding studies was conducted using affinity chromatography techniques [Liu S et al. (1999) Nature 402, 676-681; and Pfaff et al. (1998) J. Biol. Chem. 273, 6104-6109]. Recombinant GST-Paxillin (100 nM) was permitted to bind to a Ni²⁺-charged resin loaded with α4 tail protein in the absence or presence of Compound 11-A7B7C7, or the presence of Compound 11-A6B6C6 that was found to be inactive for Paxillin/α4 binding inhibition in the initial assay. Bound protein was collected and separated by electrophoresis and analyzed by immunoblotting.

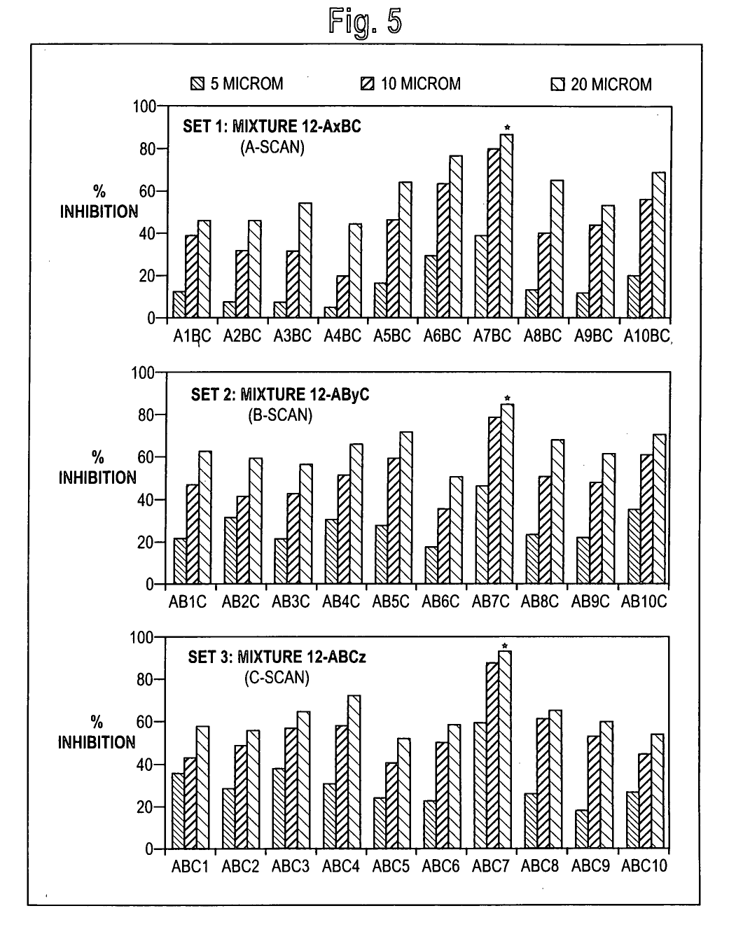

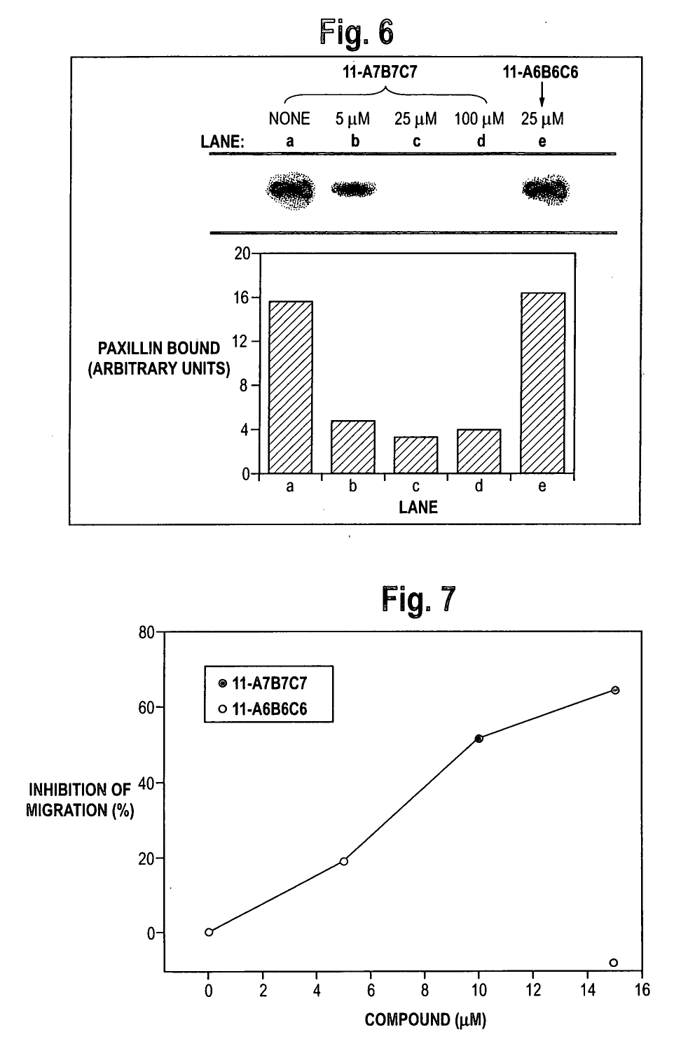

Compound 11-A7B7C7 markedly reduced the binding of Paxillin to the α4 tail protein (FIG. 5A). In contrast, Compound 11-A6B6C6 exhibited no inhibitory capacity at 25 μM. Quantification of bound Paxillin showed that Compound 11-A7B7C7 reduced Paxillin/α4 binding to near background levels at concentrations as low as 5 μM in this assay and confirmed the selectivity and effectiveness of Compound 1′-A7B7C7 at inhibiting α4/Paxillin binding.

Inhibition of Cell Migration

The functional biological activity of Compound 11-A7B7C7 was established by examining its effects on integrin α₄β₁-mediated cell migration in Jurkat T cells [Rose (2001) J. Immunol. 167, 2824-2830]. Compound 11-A7B7C7 efficiently blocked cell migration in a dose-dependent manner (IC₅₀ about 10 μM), validating the Paxillin/α4 target for therapeutic intervention. In contrast, a compound that failed to inhibit the α4/Paxillin interaction, Compound 11-A6B6C6, had no effect at concentrations up to 15 μM (FIG. 5B), the maximal concentration achievable in 1% DMSO. Compound 11-A7B7C7 did not cause leakage of cytoplasmic lactate dehydrogenase form the cells at the concentrations up to 15 μM, indicating that the inhibition of migration was not due to cytotoxicity. Furthermore, at the highest possible dose (15 μM), inhibition appeared to approach a maximum at about 70%, similar to the degree of inhibition produced by mutations in α4 that disrupt Paxillin binding.

Significance

As will be discussed further hereinafter, screening for inhibition of Paxillin binding to the integrin α4 cytoplasmic tail provided the first inhibitors of this protein-protein interaction. The lead structure emerged from a library of 1000 compounds that was prepared in two formats: (1) a traditional small mixture format composed of 100 mixtures of 10 compounds, and (2) a less traditional positional scanning library composed of larger mixtures (100 compounds/mixture). Parallel assaying of both libraries led to the identification of the same potent lead structure and provided the opportunity to critically compare the results derived from both approaches.

The deconvolution of the traditional library required resynthesis of the individual compounds from each active mixture from archived samples of the library precursors, whereas the active lead structure was deduced directly from the positional scanning testing results. The traditional library provided more and subtle SAR information regarding the Pax/α4 inhibition, whereas the positional scanning libraries provided the lead structure with considerably less synthetic and screening effort. Thus, both approaches served the purposes intended, subject to their individual limitations.

Subsequent substructure analogs of Compound 11-A7B7C7 identified structural features required for activity, those available for modification (A subunit), and those that can be removed (DMABA side chain) or modified (ester) without impacting the activity. The functional activity of Compound 11-A7B7C7 was established with its dose-dependent inhibition (IC₅₀ about 10 μM) of α₄β₁-mediated cell migration in Jurkat T cells.

Thus, complementary τo α₄β₁ antagonists that function extracellularly by inhibiting the binding of VCAM-1 or fibronectin, the intracellular inhibition of Pax/α4 binding also disrupts cell migration, offering an alternative target for therapeutic intervention by a rare example of a small molecule disruption of an intracellular protein-protein interaction [Toogood (2002) J. Med. Chem. 45, 1543-1559; Boger (2000) Helv. Chim. Acta 83, 1825-1845; Berg (2002) Proc. Natl. Acad. Sci. USA 99, 3830-3835; Boger (2001) J. Am. Chem. Soc. 123, 1280-1288; Silletti (2001) Proc. Natl. Acad. Sci. USA. 98, 119-124; Boger (2001) Bioorg. Med. Chem. 9, 557-562; and Goldberg (2002) J. Am. Chem. Soc. 124, 544-555]. Such target validation is often first established with monoclonal antibodies or peptide consensus sequences derived from endogenous ligands. It is noteworthy that the Pax/α4 target validation came from this exploration of small molecule libraries.

Experimental Procedures

Production and Purification of α4 Tail

The design and production of recombinant cytoplasmic α4 tail polypeptide have been described [Pfaff (1998) J. Biol. Chem. 273, 6104-6109]. Briefly, polymerase chain reaction was used to generate a HindIII-BamHI fragment for each wild-type or mutant integrin cytoplasmic domain. Each polymerase chain reaction product was ligated into the pCR vector using a TA cloning kit (Invitrogen). After cDNA sequencing, each fragment was ligated into HindIII-BamHI sites of the modified pET15b vector described before [Pfaff (1998) J. Biol. Chem. 273, 6104-6109]. The α4 tail was expressed in BL21(DE3)pLysS cells (Novagen), isolated by Ni²⁺-charged resins, and further purified to >90% homogeneity using a reverse-phase C18 HPLC column (Vydac).

Production and Purification of Paxillin

The expression and isolation of recombinant glutathione S-transferase (GST)-Paxillin have been described [Rose (2001) J. Immunol. 167, 2824-2830].

Immobilized Paxillin/α4 Binding Assays

Paxillin/α4 binding assays were performed as follows. Ni-NTA HisSorb microtiter strips (Qiagen) were coated overnight at 4° C. with purified α4 tail integrin (5 μg/ml, 100 μl per well) in PBS buffer (8 mM Na₂HPO₄, 1.5 mM KH₂PO₄, 135 mM NaCl, 2.5 mM KCl [pH 7.4]) supplemented with heat-denatured BSA (0.2%). The plates were washed three times with PBS buffer to remove unbound integrin, blocked with 150 μl of heat-denatured 1% BSA (denatured at 85° C. for 30 minutes and supplemented with 0.05% sodium azide) for 1 hour at room temperature, and washed again three times with PBS buffer.

Purified Paxillin in binding buffer (10% DMSO, 0.2% BSA in PBS buffer) was added to the wells at a concentration of 5 μg/ml in the presence or absence of compounds (1 mM DMSO stock solutions). During incubation, DMSO concentration never exceeded 0.5% (up to 10% DMSO alone was found to have no effect on Paxillin/α4 binding efficiency). As a control, blocked wells without integrin were examined for binding. After 1 hour at room temperature, the wells were washed three times with PBS buffer, incubated with first antibody (12CA5 ascites, 1/10000 in PBS plus 1% BSA, 100 μL) for 1 hour at room temperature, then with second antibody (100 μl HRP-conjugated anti-mouse [Biosource] in PBS buffer plus 1% BSA) for 1 hour at room temperature. Each well was treated with 50 μl of substrate solution (4 mg o-phenylenediamine, 4 μl of 30% H₂O₂, 80 mM citrate phosphate [pH 5]) and stopped after 10 minutes at room temperature with 50 μl H₂SO₄ (2 N). Binding was quantitated on a vₘₐₓ kinetic microplate reader (Molecular Devices) at 490 nm. Zero % inhibition control was measured with Paxillin and no compound, and 100% inhibition control (background) was measured with no Paxillin and no compound. Each well was duplicated and percent inhibition was averaged.

Affinity Chromatography Assay

Integrin tail affinity chromatography was performed as described [Rose (2001) J. Immunol. 167, 2824-2830]. Briefly, 1 mg of α4 integrin cytoplasmic domain dissolved in 1 ml of 8 mM Na₂HPO₄, 1.5 mM KH₂PO₄, 135 mM NaCl, 2.5 mM KCl (pH 7.4) (binding buffer) and was bound to 50 μl of Ni²⁺-charged His-Bind resin (Novagen) at 4° C. overnight (about 18 hours). The resin was then washed four times with binding buffer (1 ml) and stored in 1 ml of binding buffer at 4° C.

Soluble recombinant HA-tagged Paxillin-GST fusion protein was then added (0.5 μg) to 100 μl of integrin tail-coated resins in the presence or absence of compounds (1 mM DMSO stock solutions). The mixture was incubated at 4° C. with rotation for 1 hour. Resins were washed five times with 1 ml binding buffer. Bound proteins were extracted with 50 μl of reducing SDS sample buffer, separated on 4%-20% SDS-polyacrylamide gels (PAGE), transferred onto a nitrocellulose membrane, and analyzed by immunoblotting (anti-HA antibody [12CA5] ascites, 1/2000 for 2 hours).

Cell Migration Assay

The Jurkat E6-1 T leukemic cell line was purchased from American Type Culture Collection (ATCC), Rockville, Md. and cultured in RPMI-1640 (Biowhitaker Inc, Walkersville, Md.) supplemented with 10% FCS (Biowhitaker Inc), 1% glutamine, 50 units/ml penicillin, and 50 μg/ml streptomycin (Sigma Chemical, St Louis, Mo.).

Cell migration was assayed in a modified Boyden chamber assay system. Transwells (Costar, Corning) polycarbonate membranes containing 3.0 μm pores were incubated with VCAM-1 in 0.1 M NaHCO₃ (pH 8.0) overnight (about 18 hours) at 4° C. Membranes were blocked with 2% BSA in PBS for 30 minutes at room temperature. 2.0×10⁵ cells in RPMI-1640 with 10% FCS, 1% DMSO containing the indicated concentration of compound were added to the top chamber. SDF-1α (R&D Systems) at a final concentration of 15 ng/mL was added to the bottom chamber. Cells were permitted to migrate for 4 hours at 37° C. Cells in the bottom chamber were enumerated with a hemocytometer.

Synthetic Procedures

General procedures for the preparation of the 40 individual compounds derived from Compounds 11-AB7C6, 11-AB7C7, 11-AB7C10, and 11-AB10C9, characterization data for the four lead structures Compounds 11-A3B7C7, 11-A7B7C7, 11-A9B7C7, and 11-A7B7C6, experimental and characterization data for Compounds 19, 21-26, experimental and characterization data for Compound [¹⁴C]-11-A7B7C7 are discussed hereinbelow.

General Precedure for the Preparation of Individual Boc-AₓB₇C₇-OMe (x=3, 7, 9)

Boc-CDPI₂-OMe [Boger et al. (2000) J. Am. Chem. Soc. 122, 6382-6394; and Boger et al. (1987) J. Org. Chem. 52, 1521-1530] (450 mg, 0.9 mmol) was treated with 4 N HCl/dioxane (25 mL) for 2 hours at room temperature. The solvent was removed under a stream of N₂ and the residue was dried in vacuo overnight (about 18 hours) to afford 395 mg of a greenish solid (100% yield). The resulting crude hydrochloride salt (Compound 21, H-CDPI₂-OMe, 132 mg, 0.3 mmol, 1 equiv) was dissolved in DMF (5 mL) and treated with Boc-Aₓ-OH (0.95 equiv), followed by EDCI (2 equiv) and DMAP (2 equiv). The solutions were stirred for 48 hours at room temperature.

One-half of the DMF was removed in vacuo and the resulting suspensions were precipitated in 1 N aqueous HCl (50 mL). The compounds were isolated by centrifugation and washed using the same procedure with 1 N aqueous HCl (50 mL), then H₂O (2×50 mL). The isolated products were dried in vacuo overnight (about 18 hours) in the presence of P₂O₅.

Boc-A₃B₇C₇-OMe:

(147 mg, 77% yield); ¹H NMR (DMSO-d₆, 500 MHz) δ 11.75 (s, 1H), 11.69 (s, 1H), 8.29 (br s, 1H), 8.18 (d, 1H, J=7.4 Hz), 7.34 (d, 1H, J=8.8 Hz), 7.29 (d, 1H, J=8.8 Hz), 7.14 (s, 1H), 7.07 (s, 1H), 6.99 (s, 1H), 4.62 (t, 2H, J=8.0 Hz), 4.04 (t, 2H, J=9.2 Hz), 3.88 (s, 3H), 3.41 (t, 2H, J=8.0 Hz), 3.25 (t, 2H, J=8.1 Hz), 1.51 (s, 9H); MALDI-HRMS (DHB) m/z 649.1829 (MNa⁺, C₃₂H₃₀N₆O₆S requires 649.1840).

Boc-A₇B₇C₇-OMe (24):

(189 mg, 92% yield); ¹H NMR (DMSO-d₆, 500 MHz) δ11.97 (s, 1H), 11.77 (s, 1H), 11.71 (s, 1H), 8.29 (br s, 2H), 7.95 (d, 1H, J=8.9 Hz), 7.37 (d, 1H, J=9.9 Hz), 7.34 (d, 1H, J=8.8 Hz), 7.28 (d, 1H, J=8.9 Hz), 7.15 (s, 1H), 7.13 (s, 1H), 7.00 (s, 1H), 4.63 (m, 4H), 4.03 (t, 2H, J=8.4 Hz), 3.88 (s, 3H), 3.48-3.20 (m, 6H, partially obscured by H₂O), 1.52 (s, 9H); MALDI-HRMS (DHB) m/z 685.2800 (MH⁺, C₃₉H₃₆N₆O₆ requires 685.2769).

Boc-A₉B₇C₇-OMe:

(163 mg, 82% yield); ¹H NMR (DMSO-d₆, 500 MHz) δ 11.75 (s, 1H), 11.73 (s, 1H), 8.29 (br s, 1H), 8.21 (d, 1H, J=7.3 Hz), 7.59 (d, 1H, J=8.1 Hz), 7.42 (d, 1H, J=8.5 Hz), 7.34 (d, 1H, J=8.1 Hz), 7.28 (d, 1H, J=9.2 Hz), 7.14 (s, 1H), 7.11 (s, 1H), 6.99 (s, 1H), 6.97 (s, 1H), 4.62 (t, 2H, J=8.4 Hz), 4.02 (t, 2H, J=9.1 Hz), 3.88 (s, 3H), 3.41 (t, 2H, J=8.4 Hz), 3.25 (t, 2H, J=8.2 Hz), 1.50 (s, 9H); MALDI-HRMS (DHB) m/z 660.2464 (MH⁺, C₃₇H₃₃N₅O₇ requires 660.2453).

Preparation of Boc-A₇B₇C₆-OMe

A suspension of Boc-CDPI₂-OMe [Boger et al. (2000) J. Am. Chem. Soc. 122, 6382-6394; and Boger et al. (1987) J. Org. Chem. 52, 1521-1530] (140 mg, 0.28 mmol, 1 equiv) in 3 mL THF/MeOH/H₂O (3:2:1) was treated with LiOH.H₂O (59 mg, 1.4 mmol, 5 equiv). The reaction mixture was warmed at 50-55° C. for 24 hours. The solvent was removed under a stream of N₂, and the residual solid was resuspended in H₂O (4 mL). Aqueous HCl (1 N) was added until the mixture was acidic (pH 1-2), and the insoluble product was collected by centrifugation. The resulting solid was washed with H₂O (2×10 mL) using the same procedure. Drying the solid in vacuo (P₂O₅) afforded Boc-A₇B₇-OH (Boc-CDPI₂-OH, 136 mg, 98%) as a tan solid: ¹H NMR (DMSO-d₆, 500 MHz) δ 11.98 (s, 1H), 11.56 (s, 1H), 8.16 (br s, 1H), 7.81 (br s, 1H), 7.29 (apparent t, 2H, J=8.9 Hz), 6.99 (s, 1H), 6.79 (s, 1H), 4.61 (t, 2H, J=8.2 Hz), 4.04 (t, 2H, J=8.4 Hz), 3.98 (t, 2H, J=8.2 Hz), 3.58 (t, 2H, J=8.6 Hz), 1.52 (s, 9H); MALDI-HRMS (DHB) m/z 487.1971 (MH⁺, C₂₇H₂₆N₄O₅ requires 487.1976).

Boc-A₇B₇-OH (97 mg, 0.20 mmol, 1 equiv) was dissolved in DMF (2 mL) and treated with HCl.H—C₆-OMe (40 mg, 0.21 mmol, 0.95 equiv), followed by EDCI (77 mg, 2 equiv) and DMAP (49 mg, 2 equiv). The solutions were stirred for 22 hours at room temperature. One-half of the DMF was removed in vacuo and the resulting suspension was precipitated in 1 N aqueous HCl (50 mL). The compound was isolated by centrifugation and washed using the same procedure with 1 N aqueous HCl (25 mL), then H₂O (2×25 mL). Drying the solid in vacuo (P₂O₅) afforded Boc-A₇B₇C₆-OMe (98 mg, 79%) as a tan solid: ¹H NMR (DMSO-d₆, 500 MHz) δ11.95 (s, 1H), 10.39 (s, 1H), 8.24 (br s, 1H), 8.21 (d, 1H, J=7.9 Hz), 7.51 (s, 1H), 7.31 (d, 1H, J=8.8 Hz), 7.29 (d, 1H, J=9.1 Hz), 7.23 (s, 1H), 7.00 (s, 1H), 6.97 (s, 1H), 4.65 (t, 2H, J=8.3 Hz), 4.03 (t, 2H, J=9.0 Hz), 3.88 (s, 3H), 3.76 (s, 3H), 3.44 (t, 2H, J=8.5 Hz), 3.26 (t, 2H, J=8.1 Hz), 1.52 (s, 9H); MALDI-HRMS (DHB) m/z 623.2599 (MH⁺, C₃₄H₃₄N₆O₆ requires 623.2612).

General Precedure for the Preparation of Individual DMABA-AₓB_{y}C_{z}-OMe (11-A₃B₇C₇, 11-A₇B₇C₇, 11-A₉B₇C₇, and 11-A₇B₇C₆)

Each sample of Boc-AₓB_{y}C_{z}-OMe (1 equiv) was treated with 4 N HCl/dioxane (25 mL) for 2 hours at room temperature. The solvent was removed under a stream of N₂ and the residues were dried in vacuo overnight (about 18 hours; Compound 25 was isolated here from Boc-A₇B₇C₇-OMe, 100%). Each sample was dissolved in DMF (50 mM) was treated with 4-(dimethylamino)butyric acid (3 equiv), followed by EDCI (3 equiv) and DMAP (3 equiv). The solutions were stirred for 20 hours at room temperature. DMF was removed in vacuo and the residues were precipitated in H₂O (20 mL per 100 mg of starting material). Compounds were isolated by centrifugation and washed using the same procedure with H₂O (2×20 mL). The final products were dried in vacuo overnight (P₂O₅).

DMA-A₃B₇C₇-OMe (11-A₃B₇C₇):

(105 mg from 150 mg of starting material, 69% yield); ¹H NMR (DMSO-d₆, 500 MHz) δ 11.84 (s, 1H), 11.71 (s, 1H), 8.29 (br s, 1H), 8.20 (d, 1H, J=7.8 Hz), 7.34 (d, 1H, J=8.7 Hz), 7.29 (d, 1H, J=8.9 Hz), 7.14 (s, 1H), 7.06 (s, 1H), 6.99 (s, 1H), 4.62 (t, 2H, J=8.3 Hz), 4.19 (t, 2H, J=7.2 Hz), 4.03 (t, 2H, J=9.1 Hz), 3.88 (s, 3H), 3.41 (t, 2H, J=8.1 Hz), 3.26 (t, 2H, J=8.1 Hz), 2.79 (s, 6H), 2.59 (t, 2H, J=7.2 Hz), 1.99 (pent, 2H, J=7.0 Hz); MALDI-HRMS (DHB) m/z 662.2150 (MNa⁺, C₃₃H₃₃N₇O₅S requires 662.2156).

DMABA-A₇B₇C₇-OMe (11-A₇B₇C₇):

(175 mg from 203 mg of starting material, 85% yield); ¹H NMR (DMSO-d₆, 500 MHz) δ 11.91 (s, 1H), 11.81 (s, 1H), 11.74 (s, 1H), 8.28 (br s, 2H), 8.20 (d, 1H, J=8.8 Hz), 7.35 (d, 1H, J=8.7 Hz), 7.34 (d, 1H, J=9.4 Hz), 7.30 (d, 1H, J=8.8 Hz), 7.14 (s, 1H), 7.12 (s, 1H), 7.04 (s, 1H), 4.65 (m, 4H), 4.20 (t, 2H, J=7.2 Hz), 3.89 (s, 3H), 3.48-3.20 (m, 6H, partially obscured by H₂O), 3.11 (t, 2H, J=8.2 Hz), 2.76 (s, 6H), 2.58 (t, 2H, J=7.0 Hz), 1.95 (pent, 2H, J=7.0 Hz); MALDI-HRMS (DHB) m/z 698.3073 (MH⁺, C₄₀H₃₉N₇O₅ requires 698.3085).

DMABA-A₉B₇C₇-OMe (11-A₉B₇C₇):

(138 mg from 171 mg of starting material, 79% yield); ¹H NMR (DMSO-d₆, 500 MHz) δ 11.81 (s, 1H), 11.69 (s, 1H), 8.30 (br s, 1H), 8.20 (d, 1H, J=7.5 Hz), 7.64 (d, 1H, J=8.1 Hz), 7.57 (d, 1H, J=8.9 Hz), 7.33 (d, 1H, J=8.2 Hz), 7.28 (d, 1H, J=8.9 Hz), 7.14 (s, 1H), 7.11 (s, 1H), 7.03 (s, 1H), 6.99 (s, 1H), 4.62 (t, 2H, J=8.4 Hz), 4.18 (t, 2H, J=7.3 Hz), 4.02 (t, 2H, J=8.9 Hz), 3.88 (s, 3H), 3.41 (t, 2H, J=8.1 Hz), 3.25 (t, 2H, J=8.2 Hz), 2.74 (s, 6H), 2.54 (t, 2H, J=7.3 Hz), 1.96 (pent, 2H, J=7.1 Hz); MALDI-HRMS (DHB) m/z 673.2772 (MH⁺, C₃₈H₃₆N₆O₆ requires 673.2769).

DMABA-A₇B₇C₆-OMe (11-A₇B₇C₆):

(69 mg from 94 mg of starting material, 72% yield); ¹H NMR (DMSO-d₆, 500 MHz) δ 11.93 (s, 1H), 10.73 (s, 1H), 8.24 (br s, 1H), 8.20 (d, 1H, J=7.7 Hz), 7.52 (s, 1H), 7.35 (d, 1H, J=8.4 Hz), 7.29 (d, 1H, J=8.2 Hz), 7.25 (s, 1H), 7.03 (s, 1H), 6.99 (s, 1H), 4.65 (t, 2H, J=8.2 Hz), 4.20 (t, 2H, J=8.8 Hz), 3.88 (s, 3H), 3.76 (s, 3H), 3.59 (t, 2H, J=7.2 Hz), 3.44 (t, 2H, J=8.3 Hz), 3.28 (t, 2H, J=8.1 Hz), 2.81 (t, 2H, J=7.4 Hz), 2.56 (s, 6H), 1.89 (pent, 2H, J=7.2 Hz); MALDI-HRMS (DHB) m/z 636.2931 (MH⁺, C₃₅H₃₇N₇O₅ requires 636.2929).

DMABA-CDPI-OMe (Compound 19):

H-CDPI-OMe (Compound 16, 10 mg, 46.2 μmol, 1 equiv) was dissolved in DMF (600 μL) and the resulting solution was treated with 4-(dimethylamino)butyric acid (15.5 mg, 2 equiv), followed by EDCI (17.8 mg, 2 equiv) and DMAP (11.3 mg, 2 equiv). The solution was stirred for 20 hours at room temperature, before the DMF was removed in vacuo. The residue was suspended in H₂O (1 mL) and the aqueous solution was extracted with ethyl acetate (EtOAc) (4×2 mL). The organic extracts were pooled, dried (Na₂SO₄), filtered and concentrated in vacuo (P₂O₅) to afford 11.1 mg (75%) of DMABA-CDPI-OMe (19): ¹H NMR (CDCl₃, 500 MHz) δ8.88 (br s, 1H), 7.35 (br s, 1H), 7.23 (br s, 1H), 7.01 (br s, 1H), 4.36 (t, 2H, J=9.0 Hz), 3.92 (s, 3H), 3.22 (t, 2H, J=9.2 Hz), 2.87 (t, 2H, J=7.4 Hz), 2.66 (s, 6H), 2.57 (t, 2H, J=7.3 Hz), 1.86 (pent, 2H, J=7.1 Hz); MALDI-HRMS (DHB) m/z 330.1817 (MH⁺, C₁₈H₂₃N₃O₃ requires 330.1822).

DMABA-CDPI₂-OMe (Compound 22):

H-CDPI₂-OMe (Compound 21, 8 mg, 18.3 μmole, 1 equiv) was dissolved in DMF (360 μL) and the resulting solution was treated with 4-(dimethylamino)butyric acid (9.2 mg, 3 equiv), followed by EDCI (10.5 mg, 3 equiv) and DMAP (6.7 mg, 3 equiv). The solution was stirred for 20 hours at room temperature. DMF was removed in vacuo and the residue was precipitated in H₂O (1 mL). The compound was isolated by centrifugation and washed using the same procedure with H₂O (2×1 mL). The final product was dried in vacuo (P₂O₅) to afford 8.4 mg (90%) of DMABA-CDPI₂-OMe (22): ¹H NMR (DMSO-d₆, 500 MHz) δ 11.75 (s, 1H), 10.94 (s, 1H), 8.29 (br s, 1H), 8.18 (d, 1H, J=9.0 Hz), 7.33 (d, 1H, J=8.4 Hz), 7.27 (d, 1H, J=9.1 Hz), 7.17 (s, 1H), 7.01 (s, 1H), 4.62 (t, 2H, J=8.1 Hz), 4.02 (t, 2H, J=7.9 Hz), 3.89 (s, 3H), 3.41 (t, 2H, J=8.2 Hz), 3.29 (t, 2H, J=8.0 Hz), 2.87 (t, 2H, J=7.4 Hz), 2.74 (s, 6H), 2.57 (t, 2H, J=7.3 Hz), 1.95 (pent, 2H, J=7.0 Hz); MALDI-HRMS (DHB) m/z 514.2463 (MH⁺, C₂₉H₃₁N₅O₄ requires 514.2459).

H-CDPI₂-OH (Compound 23):

H-CDPI₂-OMe (Compound 21, 5 mg, 11.45 μmole, 1 equiv) in 3 mL THF/MeOH (2:1) was treated with 0.055 N aqueous LiOH (1 mL, 5 equiv). The reaction mixture was warmed at 45-50° C. for 20 hours. The solvent was removed under a stream of N₂, and the residual solid was resuspended in aqueous 1 N aqueous HCl (2 mL, pH 1-2), and the insoluble product was collected by centrifugation. The resulting solid was washed with H₂O (2×1 mL) using the same procedure. Drying the solid in vacuo (P₂O₅) afforded H-CDPI₂-OH (3.3 mg, 75%) as a tan solid: ¹H NMR (DMSO-d₆, 500 MHz) δ 11.64 (s, 1H), 11.23 (s, 1H), 8.54 (br s, 1H), 8.28 (d, 1H, J=9.0 Hz), 7.26 (apparent t, 2H, J=8.9 Hz), 7.15 (s, 1H), 6.92 (s, 1H), 4.37 (t, 2H, J=7.9 Hz), 4.03 (t, 2H, J=8.2 Hz), 3.42 (t, 2H, J=8.4 Hz, partially obscured by H₂O), 3.22 (t, 2H, J=8.0 Hz); MALDI-HRMS (DHB) m/z 387.1454 (MH⁺, C₂₂H₁₈N₄O₃ requires 387.1462).

H-CDPI₃-OH (Compound 26):

H-CDPI₃-OMe (Compound 25, 5 mg, 8.05 μmole, 1 equiv) in 3 mL THF/MeOH (2:1) was treated with 0.33 N aqueous LiOH (1 mL, 30 equiv). The reaction mixture was warmed at 55-60° C. for 20 hours. The solvent was removed under a stream of N₂, and the residual solid was resuspended in aqueous 1 N aqueous HCl (2 mL, pH 1-2), and the insoluble product was collected by centrifugation. The resulting solid was washed with H₂O (2×1 mL) using the same procedure. Drying the solid in vacuo (P₂O₅) afforded H-CDPI₃-OH (4.3 mg, 94%) as a tan solid: ¹H NMR (DMSO-d₆, 500 MHz) δ11.99 (s, 1H), 11.74 (s, 1H), 11.64 (s, 1H), 8.67 (br s, 1H), 8.32 (br s, 1H), 7.96 (d, 1H, J=8.6 Hz), 7.35 (d, 1H, J=9.3 Hz), 7.31 (d, 1H, J=8.5 Hz), 7.21 (d, 1H, J=8.2 Hz), 7.15 (s, 1H), 6.98 (s, 1H), 6.94 (s, 1H), 4.29 (m, 4H), 4.13 (t, 2H, J=8.2 Hz), 3.98-3.52 (m, 4H, partially obscured by H₂O), 3.29 (t, 2H, J=8.1 Hz); MALDI-HRMS (DHB) m/z 571.2103 (MH⁺, C₃₃H₂₆N₆O₄ requires 571.2098).

[¹⁴C]-11-A₇B₇C₇

A suspension of 11-A₇B₇C₇ (DMABA-A₇B₇C₇-OMe; 20 mg, 28.6 μmole, 1 equiv) in 1.15 mL THF/MeOH (3:2) was treated with 4 N aqueous LiOH (220 μL, 30 equiv). The reaction mixture was warmed at 55-60° C. for 48 hours. The solvent was removed under a stream of N₂, the residual solid was resuspended in aqueous 1 N aqueous HCl (2 mL, pH 1-2), and the insoluble product was collected by centrifugation. The resulting solid was washed with H₂O (2 mL) using the same procedure. Drying the solid in vacuo (P₂O₅) afforded DMABA-A₇B₇C₇-OH (16.8 mg, 86% yield) as a tan solid: ¹H NMR (DMSO-d₆, 500 MHz) δ 11.95 (s, 1H), 11.74 (s, 1H), 9.88 (s, 1H), 8.27 (br s, 2H), 8.21 (d, 1H, J=8.6 Hz), 7.32 (d, 1H, J=8.4 Hz), 7.29 (d, 1H, J=9.1 Hz), 7.24 (d, 1H, J=8.4 Hz), 7.11 (s, 1H), 7.06 (s, 1H), 7.04 (s, 1H), 4.64 (m, 4H), 4.20 (t, 2H, J=7.3 Hz), 4.01 (t, 2H, J=8.4 Hz), 3.52-3.18 (m, 4H, partially obscured by H₂O), 3.11 (t, 2H, J=8.1 Hz), 2.79 (s, 6H), 2.60 (t, 2H, J=7.2 Hz), 1.97 (pent, 2H, J=7.2 Hz); MALDI-HRMS (DHB) m/z 684.2930 (MH⁺, C₃₉H₃₇N₇O₅ requires 684.2929).

A suspension of DMABA-A₇B₇C₇-OH (6.0 mg, 8.71 μmol, 1 equiv), EDCI (5.0 mg, 26.1 μmole, 3 equiv), and DMAP (2.12 mg, 17.4 μmol, 2 equiv) was treated with ¹⁴CH₃OH (Sigma, 0.5 mCi, 41 mCi/mmol, 1.4 equiv) and the mixture was stirred at room temperature for 20 hours. The solvent was cautiously removed under a stream of N₂, and the residual solid was precipitated in H₂O (1 mL). The suspension was centrifuged and the solid was washed using the same procedure with H₂O (2×1 mL). The final product was dried in vacuo overnight (about 18 hours; P₂O₅) to afford 4.5 mg (74% yield) of [¹⁴C]-11-A₇B₇C₇ as a yellow solid. This material was identical with all respects to unlabeled 11-A₇B₇C₇ by ¹H NMR. TLC (EtOAc/Hexane/Et₃N=60:30:5 showed a single radioactive spot that co-migrated with unlabeled 11-A₇B₇C₇. Specific activity was calculated to be identical to that of the radioactive precursor (¹⁴CH₃OH: 41 mCi/mmol).

Each of the patents, applications and articles cited herein is incorporated by reference. The use of the article “a” or “an” is intended to include one or more.

From the foregoing it will be observed that numerous modifications and variations can be effectuated without departing from the true spirit and scope of the novel concepts of the invention. It is to be understood that no limitation with respect to the specific embodiment illustrated is intended or should be inferred. The disclosure is intended to cover by the appended claims all such modifications as fall within the scope of the claims.

What is claimed:

1. A purified compound whose structure corresponds to Formula I, below, or its pharmaceutically acceptable salt 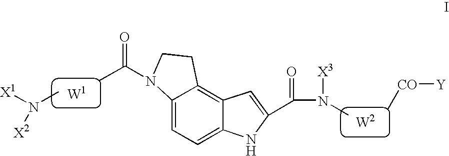
wherein the structures W¹ and W² are the same or different and are an aromatic ring system containing one, two or three five-, six- or seven-membered rings of which at least one is aromatic,
X¹ and X² are independently hydrido or R¹—C(O), R¹—NHC(O), or R¹—NHC(S), and R¹ is hydrido or ZQ, or X² is a bond between the depicted nitrogen atom and the structure W¹ so that the depicted nitrogen atom is a ring atom of structure W¹;
X³ is hydrido or is a bond between the depicted nitrogen atom and the structure W² so that the depicted nitrogen atom is a ring atom of structure W²;
Z is amino wherein the amino nitrogen is (i) unsubstituted, or (ii) substituted with one or two substituents containing a total of up to fourteen, atoms that are carbon, nitrogen, oxygen or sulfur that are independently selected from the group consisting of an alkyl, alkenyl, alkynyl, aryl, heteroaryl, aralkyl, cycloalkyl, heterocyclo, and a heterocycloalkyl group, group, or (iii) wherein the amino nitrogen and two substituents attached thereto form a 5- to 8-membered heterocyclo or heteroaryl ring containing zero to two additional heteroatoms that are nitrogen, oxygen or sulfur, and
Q is a hydrocarbyl group containing two to about ten carbon atoms; and
Y is O—R² or Z, wherein R² is hydrido, methyl or Q.

2. The purified compound according to claim 1 wherein one or both of W¹ and W² contains at least two fused rings.

3. The purified compound according to claim 1 wherein one or both of W¹ and W² contains three fused rings.

4. The purified compound according to claim 1 wherein one or both of W¹ and W² contains a ring atom a heteroatom that is oxygen, sulfur or nitrogen.

5. The purified compound according to claim 1 whose structure corresponds to Formula II, below, or its pharmaceutically acceptable salt 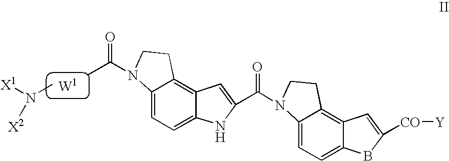
wherein B is O, NH or S, and each of W¹, X¹, X² and Y is as defined before.

6. The purified compound according to claim 5 wherein B is NH.

7. The purified compound according to claim 6 wherein W¹ contains at least two fused rings.

8. The purified compound according to claim 7 wherein W¹ contains three fused rings.

9. The purified compound according to claim 8 wherein X² is a bond between the depicted nitrogen atom and the structure W¹ so that the depicted nitrogen atom is a ring atom of structure W¹

10. A purified compound whose structure corresponds to Formula III, below, or its pharmaceutically acceptable salt 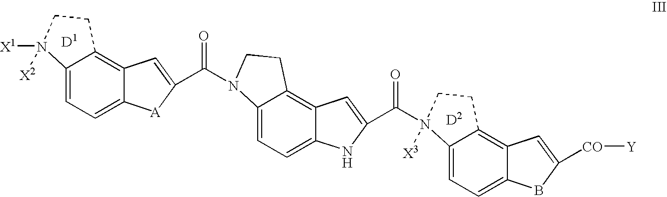
wherein each of A and B is independently O, NH or S;
dotted portions of structures D¹ and D² are independently present or absent such that when either is present, the corresponding X² and X³ substituent is absent;
X¹ and X², when present, are independently hydrido or R¹—C(O), R¹—NHC(O), or R¹—NHC(S), and R¹ is hydrido or ZQ, or when D¹ is present, X² is a bond between the depicted nitrogen atom and the dotted ring structure so that the depicted nitrogen atom is a ring atom of the dotted structure D¹;
X³ is hydrido or is a bond between the depicted nitrogen atom and the dotted structure so that the depicted nitrogen atom is a ring atom of the dotted structure D²;
Z is amino wherein the amino nitrogen is (i) unsubstituted, or (ii) substituted with one or two substituents containing a total of up to fourteen atoms that are carbon, nitrogen, oxygen or sulfur that are independently selected from the group consisting of an alkyl, alkenyl, alkynyl, aryl, heteroaryl, aralkyl, cycloalkyl, heterocyclo, and a heterocycloalkyl group, group, or (iii) wherein the amino nitrogen and two substituents attached thereto form a 5- to 8-membered heterocyclo or heteroaryl ring containing zero to two additional heteroatoms that are nitrogen, oxygen or sulfur, and
Q is a hydrocarbyl group containing two to about ten carbon atoms; and
Y is O—R² or Z, wherein R² is hydrido, methyl or Q.

11. The purified compound according to claim 10 wherein at least one of D¹ and D² is present, at least one of X² and X³ is a bond between the depicted nitrogen atom and the dotted ring structure, so that at least one of the depicted nitrogen atoms is a ring atom of the dotted structure D¹ or the dotted structure D².

12. The purified compound according to claim 11 wherein both of D¹ and D² are present.

13. The purified compound according to claim 12 wherein at least one of A and B is NH.

14. The purified compound according to claim 10 wherein at least one of A and B is NH.

15. A purified compound whose structure corresponds to Formula IV, or a pharmaceutically acceptable salt thereof 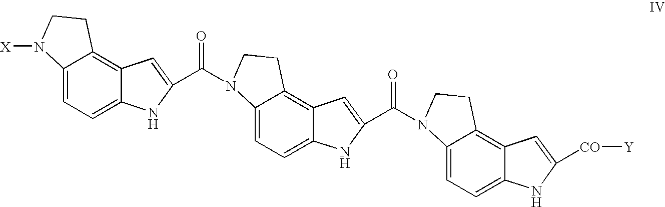
wherein X is hydrido or R¹—C(O), R¹—NHC(O), or R¹—NHC(S), and R¹ is hydrido or ZQ,
Z is amino wherein the amino nitrogen is (i) unsubstituted, or (ii) substituted with one or two substituents containing a total of up to fourteen atoms that are carbon, nitrogen, oxygen or sulfur and which substituents are independently selected from the group consisting of an alkyl, aryl, heteroaryl, aralkyl, cycloalkyl, aralkoxycarbonyl, alkoxycarbonyl, arylcarbonyl, aralkanoyl, heteroarylcarbonyl and an alkanoyl group, or (iii) wherein the amino nitrogen and two substituents attached thereto form a 5- to 8-membered heterocyclo or heteroaryl ring containing zero to two additional heteroatoms that are nitrogen, oxygen or sulfur, and
Q is a hydrocarbyl group containing two to about ten carbon atoms; and
Y is O—R² or Z,
wherein R² is hydrido, methyl or Q.

16. The purified compound according to claim 15 that corresponds in structure to Formula A, or a pharmaceutically acceptable salt thereof

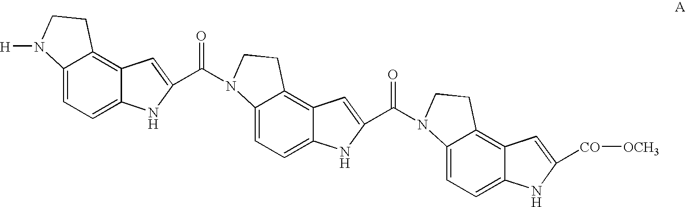

17. The purified compound according to claim 15 that corresponds in structure to Formula B, or a pharmaceutically acceptable salt thereof

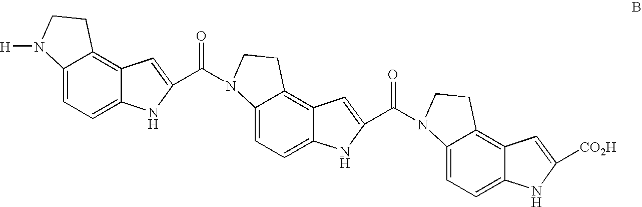

18. The purified compound according to claim 16 that corresponds in structure to Formula C, or a pharmaceutically acceptable salt thereof

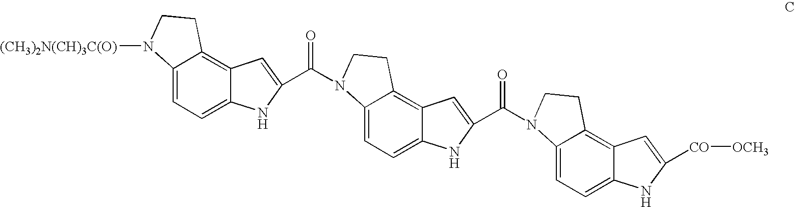

19. A pharmaceutical composition that contains an effective amount of a purified compound or its salt of claim 1 dissolved or dispersed in a physiologically acceptable diluent.

20. A pharmaceutical composition that contains an effective amount of a purified compound or its salt of claim 5 dissolved or dispersed in a physiologically acceptable diluent.

21. A pharmaceutical composition that contains an effective amount of a purified compound or its salt of claim 10 dissolved or dispersed in a physiologically acceptable diluent.

22. A pharmaceutical composition that contains an effective amount of a purified compound or its salt of claim 15 dissolved or dispersed in a physiologically acceptable diluent.

23. A method of treating a biological function in an animal that is mediated by integrin/Paxillin binding that comprises administering to a mammal in need thereof an effective amount of a compound that corresponds in structure to Formula I or a pharmaceutically acceptable salt of said compound dissolved or dispersed in a physiologically acceptable diluent 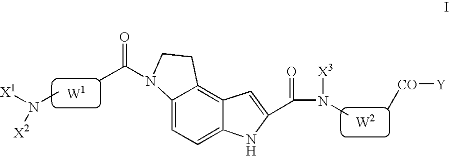
wherein the structures W¹ and W² are the same or different and are an aromatic ring system containing one, two or three five-, six- or seven-membered rings of which at least one is aromatic,
X¹ and X² are independently hydrido or R¹—C(O), R¹—NHC(O), or R¹—NHC(S), and R¹ is hydrido or ZQ, or X² is a bond between the depicted nitrogen atom and the structure W¹ so that the depicted nitrogen atom is a ring atom of structure W¹;
X³ is hydrido or is a bond between the depicted nitrogen atom and the structure W² so that the depicted nitrogen atom is a ring atom of structure W²;
Z is amino wherein the amino nitrogen is (i) unsubstituted, or (ii) substituted with one or two substituents containing a total of up to fourteen atoms that are carbon, nitrogen, oxygen or sulfur that are independently selected from the group consisting of an alkyl, alkenyl, alkynyl, aryl, heteroaryl, aralkyl, cycloalkyl, heterocyclo, and a heterocycloalkyl group, group, or (iii) wherein the amino nitrogen and two substituents attached thereto form a 5- to 8-membered heterocyclo or heteroaryl ring containing zero to two additional heteroatoms that are nitrogen, oxygen or sulfur, and
Q is a hydrocarbyl group containing two to about ten carbon atoms; and
Y is O—R² or Z, wherein R² is hydrido, methyl or Q.

24. The method according to claim 23 wherein said biological function is inflammation.

25. The method according to claim 23 wherein said biological function is scarring during wound healing.

26. The method according to claim 23 wherein said biological function is atherosclerosis.

27. The method according to claim 23 wherein said compound corresponds in structure to Formula II, or a pharmaceutically acceptable salt thereof 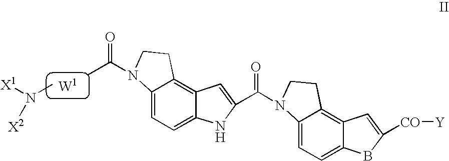
wherein B is O, NH or S, and each of W¹, X¹, X² and Y is as defined before.

28. The method according to claim 23 wherein said compound corresponds in structure to Formula III, or a pharmaceutically acceptable salt thereof 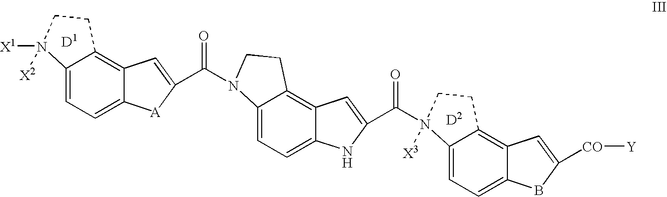
wherein each of A and B is independently O, NH or S;
dotted portions of structures D¹ and D² are independently present or absent such that when either is present, the corresponding X² and X³ substituent is absent;
X¹ and X², when present, are independently hydrido or R¹—C(O), R¹—NHC(O), or R¹—NHC(S), and R¹ is hydrido or ZQ, or when D¹ is present, X² is a bond between the depicted nitrogen atom and the dotted ring structure so that the depicted nitrogen atom is a ring atom of the dotted structure D¹;
X³ is hydrido or is a bond between the depicted nitrogen atom and the dotted structure so that the depicted nitrogen atom is a ring atom of the dotted structure D²;
Z is amino wherein the amino nitrogen is (i) unsubstituted, or (ii) substituted with one or two substituents containing a total of up to fourteen atoms that are carbon, nitrogen, oxygen or sulfur that are independently selected from the group consisting of an alkyl, alkenyl, alkynyl, aryl, heteroaryl, aralkyl, cycloalkyl, heterocyclo, and a heterocycloalkyl group, group, or (iii) wherein the amino nitrogen and two substituents attached thereto form a 5- to 8-membered heterocyclo or heteroaryl ring containing zero to two additional heteroatoms that are nitrogen, oxygen or sulfur, and
Q, Y and Z are as defined before.

29. The method according to claim 23 wherein said animal is a mammal.

30. The method according to claim 23 wherein said animal is a reptile.

31. The method according to claim 23 wherein said integrin is the α4 integrin.
